SEQUENCE LISTING (8 sequences)

SEQ ID NO: 1 - protein, 20 aa, unidentified
CCCTCGTTTTGCCATGAACC

SEQ ID NO: 2 - protein, 20 aa, unidentified
CCAGTGTTCCAAGGTCCACA

SEQ ID NO: 3 - protein, 22 aa, unidentified
ACATTGCTGCACAAGGTCCCAG

SEQ ID NO: 4 - protein, 22 aa, unidentified
AACCATCAGGCATCTCTGTCGC

SEQ ID NO: 5 - protein, 19 aa, unidentified
ATTCCTATGTGGGCGACGA

SEQ ID NO: 6 - protein, 19 aa, unidentified
AATGGTGATGACCTGGCCG

SEQ ID NO: 7 - protein, 25 aa, unidentified
GATCTTCCCCATCGGTGATGTCGGC

SEQ ID NO: 8 - protein, 22 aa, unidentified
GCTGGCAAGTGTAGCGGTCACG

(19) United States
(12) Patent Application Publication
(10) Pub. No.: US 2025/0129418 A1
(43) Pub. Date: Apr. 24, 2025

(54) MICROCAPSULES COMPRISING BIOLOGICAL SAMPLES, AND METHODS FOR USE OF SAME

(71) Applicant: VILNIUS UNIVERSITY, Vilnius (LT)

(72) Inventors: Linas MAZUTIS, Vilnius (LT); Greta LEONAVICIENE, Vilnius (LT)

(21) Appl. No.: 18/715,033

(22) PCT Filed: Dec. 1, 2022
(86) PCT No.: PCT/EP2022/084063

§ 371 Date: May 30, 2024

Related U.S. Application Data
(60) Provisional application No. 63/284,662, filed on Dec. 1, 2021.

(51) Int. Cl. C12Q 1/6876; C12Q 1/6811; C12Q 1/6851

(52) U.S. Cl. CPC C12Q 1/6876; C12Q 1/6811; C12Q 1/6851; C12Q 2600/156; C12Q 2600/158

(57) ABSTRACT

The present invention concerns a method of performing one or more reactions on a biological entity, the method comprising: (i) isolating the biological entity in a microcapsule comprising a core and a semi-permeable shell; and (ii) performing the one or more reactions on the biological entity in the microcapsule.

### FUNDING

Part of this invention was made with funding supported under Grant No. 01.2.2-LMT-K-718-04-0002 awarded by the Research Council of Lithuania.

### TECHNICAL FIELD

The present invention concerns microcapsules comprising a semi-permeable shell and a core, which encapsulate biological entities/species (e.g. cells, bacteria, viruses, nucleic acids, biochemical compounds) and/or other material. The invention further provides the use of the microcapsules in methods, particularly multi-step methods, performed on the biological entities/species. Preferred methods involved single-cell encapsulation and analysis of nucleic acids obtained from the cell. The invention also provides methods for producing the microcapsule encapsulating biological entities, and kits suitable for producing the microcapsules.

### BACKGROUND OF INVENTION

Single-cell genomics studies are important for life sciences and biomedicine. Single-cell reverse transcription polymerase chain reaction (RT-PCR), single-cell PCR, single-cell genome amplification and other single-cell genomics assays could facilitate cancer diagnostics, identification of circulating cells in bodily fluids, characterizing genetic heterogeneity or copy number variation amongst others.

A variety of methods have been developed to isolate individual cells, amplify and analyze their genetic material [1, 2], including: US20100092973A1, which reports purified nucleic acid reaction using “droplet-in-oil” technology (but does not reveal the use of capsules for single-cell RT-PCR); Chen et al., [3] which reports the use of polysaccharide-based capsules based on alginate and dextran to isolate cells but, however, does not prove that the reported capsules are compatible with nucleic acid amplification and analysis assays; and Zhu et al, [4] which reports that the target DNA of interest can be amplified using the hydrogel (agarose) beads. In particular, Zhu et al., showed that liquid agarose droplets having bacteria cells can be gelated into solid beads with the fluorescently labelled PCR amplicons covalently attached to the agarose mesh. Each agarose droplet contained two forward primers labelled with FAM or Cy5 fluorophore targeting region of interest (K12 or O157 respectively), while two types of reverse primers are covalently conjugated to agarose matrix. The solidified beads were washed to remove the fluorescently labelled primers that were not used during PCR reaction and beads were then analyzed using fluorescence microscopy or flow cytometry.

Most of the single-cell analytical methods reported to-date rely on homogenous reaction conditions, where cell lysis and nucleic acid amplification reaction are performed in the same reaction mix without addition of new reagents, or without replacing old reagents. Because nucleic acid amplification and cell lysis are conducted in the same reaction mix, the ability to efficiently lyse the encapsulated cells (e.g. using harsh lysis reagents such as SDS, or guanidinium thiocyanate) becomes limited. In addition, some lysis agents can inhibit nucleic acid molecule amplification and processing. Therefore, in single-cell RT and single-cell RT-PCR assays the cell lysis is typically performed under mild conditions, using non-ionic detergents that are compatible with reverse transcriptase, DNA/RNA polymerase enzymes. Moreover, the nucleic acid amplification reaction inhibition by the cell lysate and/or cell debris pose a significant drawback for single-cell RT-PCR and single-cell PCR assays. As was shown previously RT-PCR inhibition becomes significant at concentrations of 1 cell in 5 nanoliter volume [5]. While some groups have shown that it is possible to achieve single-step RT-PCR on single-cells in nano-wells having a volume in the range of 100-200 pl, the results were highly variable even for highly expressed gene, and generally suffered from reduced sensitivity [6]. To overcome cell lysate-mediated inhibition of RT-PCR, others have introduced complex microfluidic workflows, to first encapsulate and lyse individual cells in one compartment (droplet) and then dilute the resultant lysate compartment 20-fold by performing controlled droplet fusion followed by cell lysate compartment splitting and second fusion (picoinjection) with PCR reagents and TaqMan probes [7]. The drawbacks of such approaches are that a fraction of lysate is lost due during droplet splitting and entire workflow relies on complex microfluidic operations on-chip and off-chip that are not readily available to majority of researchers.

Using similar approach, Pellegrino et al., applied a simplified, two-step microfluidic workflow to perform multi-step RT-PCR reaction on single-cells. After cell encapsulation and lysis using 1^{st} microfluidics chip the droplets containing the cell lysates are reinjected into a 2^{nd} microfluidics chip and merged with a second droplet containing PCR reagents and hydrogel beads carrying covalently attached PCR primers. After co-encapsulation of the hydrogel beads with the droplets containing cell lysate, the PCR primers are photo-released and the target genomic DNA locus is amplified by means of PCR and sequenced [8]. The drawback of such approach is reliance on two-step droplet manipulations on-chip and off-chip that are difficult to perform, require careful emulsion handling and can cause uncontrolled droplet coalescence. In addition, the use hydrogel beads to deliver PCR primers can reduce cell and bead co-encapsulation events, and thus as a result fraction of cells will not be amplified during PCR step. Finally, the method suffers from reduced analytical sensitivity (e.g. occurrence of allelic dropouts).

Considering the difficulties associated with emulsion handling during multi-step and two-step procedures, Kim et al., introduced a system for performing all droplet operations on a single, integrated microfluidic chip. Such integrated system enables cell encapsulation with lysis buffer, reagent mixing, oil withdrawal, droplet packing and incubation in a delay line, droplet containing a cell lysate fusion with RT-PCR reagents and finally nucleic acid amplification and readout using TaqMan probes [9]. However, such workflow relies on a sophisticated microfluidics system that is difficult to implement and control, while the expertise needed to operate the system is not readily available outside specialized laboratories.

To overcome complex off-chip and on-chip droplet manipulation workflows needed for single-cell PCR and single-cell RT-PCR assay, others have attempted to make of use hydrogel beads, where the individual cells are isolated and embedded into a hydrogel mesh. The hydrogel beads are permeable to small molecules, such as enzymes and detergents, but can trap larger biomolecules, such as genomic DNA. As a result, hydrogel beads carrying cells can be processed through sequential, multistep reactions (e.g. comprising cell lysis and subsequent washing steps, and other). The noticeable examples of hydrogel-bead systems include single-cell genotypic assays [10-12] and screening [13].

However, there are major drawbacks of the hydrogel-bead based methods that limit their use for single-cell PCR and single-cell RT-PCR applications. First, during hydrogel bead generation and physical gelation and/or covalent cross-linking reaction, a fraction of encapsulated cells might be lost because cells tend to sediment, and/or adhere to the water-oil interface [14]. Second, when hydrogel-bead embedded cells are trapped near the edge the gDNA released during the lysis step becomes susceptible to diffusion out of the hydrogel mesh, and thus eventual loss. Third, the uniform permeability of the hydrogel mesh precludes the selective retention and exchange of reagents allowing only relatively large biomolecules to be retained inside the hydrogel. For example, as it was shown by Rakszewska et al., and Zhang et al., to retain mRNA molecules in a cell lysate, the hydrogel mesh needs to be functionalized with DNA oligonucleotides [10, 11]. Therefore, the copy DNA and/or PCR products are covalently attached to the hydrogel mesh. Fourth, the biochemical reaction efficiency and sensitivity can be affected by steric hindrance and reduced diffusivity inside hydrogel mesh [15-18] or undesirable electrostatic interactions with the hydrogel mesh [19, 20]. Fifth, in some state-of-the-art examples the hydrogel bead embedded cells needs to be re-encapsulated in microfluidic droplets or fused with 2^{nd} set of droplets containing nucleic acid amplification/analysis reagents [12]. Such approach increases the overall complexity of a workflow as does the droplet-based microfluidic approaches discussed above.

Therefore, the state-of-the-art techniques and methods show that while it is possible to perform single-cell PCR or single-cell RT-PCR assays in reaction volumes below 1 nanoliter, yet the existing approaches are either very complex and involve sophisticated microfluidic chip(s) or system(s), rely on sophisticated multi-step droplet manipulation procedures on-chip, or as in hydrogel bead examples, are prone to cell and/or nuclei acid loss, reduced sensitivity and other analytical and technical drawbacks. We postulated that micro-compartments surrounded by semi-permeable shell should mitigate aforementioned technical and analytical constrains and pave the way for simple and efficient single-cell PCR and single-cell RT-PCR assays to be conducted in a massively parallel fashion.

To this aim, we have recently reported a method for production of the semi-permeable capsules, composed of polyethylene glycol diacrylate (PEGDA) and dextran [14], (and US 2020/0400538 and WO 2020/255108). In the published work we showed examples of the use of the semi-permeable capsules for genotypic and phenotypic analysis of individual bacterial cells by performing multi-step workflows on hundreds and thousands of cells simultaneously. However, capsules approaching 50-60 μm size were more difficult to produce and they tended to lose the concentricity. This may limit their ease of use in some particular biological assays that rely on isolation of individual mammalian cells where micro-compartments (e.g. water droplets) that are close to 50 μm in diameter, or beyond, may be required. Moreover, since the PEGDA monomers are partly soluble in the dextran phase, upon polymerization the microcapsule's core forms a covalently crosslinked hydrogel mesh and cannot be converted back to a liquid form. Therefore, said microcapsules should be considered as a core-shell microparticle composed on two hydrogel layers; hydrogel constituting the inner core of the microcapsule and rich in dextran, and hydrogel constituting the outer shell of the microcapsule and rich in PEGDA. This may restrict their utility in some cases.

The presence of hydrogel core in some cases may reduce enzymatic reaction efficiency and sensitivity. For example, steric hindrance and reduced reagent and/or substrate diffusivity due to hydrogel mesh and undesirable electrostatic interactions with the hydrogel mesh may affect enzymatic reaction efficiency and sensitivity.

Accordingly, there remains a need to provide alternative options and improvements to the methods and microcapsules previously described in the art, in particular in order to extend their applicability in the analysis of biological samples and increase their ease of use.

### SUMMARY OF THE INVENTION

According to a first aspect described herein there is provided a microcapsule comprising:
(a) a core;
(b) a semi-permeable shell surrounding the core; and
(c) a biological entity in the core,
   the core comprising an antichaotropic agent and/or a polyhydroxy compound, and
   the semi-permeable shell comprising a gel formed from a polyampholyte, wherein the polyampholyte in the gel is covalently cross-linked.

In a second aspect the present invention provides a plurality of microcapsules of the first aspect, wherein at least 1% of the plurality of microcapsule comprise a single cell, preferably wherein at least 10% of the plurality of microcapsules comprise a single cell.

In a third aspect the present invention provides a composition comprising a microcapsule according to the first aspect, or a plurality of microcapsule according to the second aspect, in a carrier oil or an aqueous solution.

In a fourth aspect the present invention provides a method of producing a microcapsule encapsulating a biological entity according to the first aspect, the method comprising:
(a) forming a water-in-oil droplet comprising a first solute, a second solute and the biological entity, wherein the first solute is a polyampholyte and the second solute is an antichaotropic agent and/or a polyhydroxy compound, wherein the polyampholyte comprises one or more covalently cross-linkable groups;
(b) allowing aqueous phase separation inside the water-in-oil droplet into a shell phase enriched in the first solute and a core phase enriched in the second solute, and gelation and/or precipitation of the shell phase to form an intermediate microcapsule with a solidified shell;
(c) forming intermolecular covalent cross-links with the one or more covalently cross-linkable groups to form the microcapsule comprising a semi-permeable shell of covalently cross-linked polyampholyte and a core, wherein the biological entity is in the core.

In a fifth aspect the present invention provides a method of performing one or more reactions on a biological entity, the method comprising:
(i) isolating the biological entity in a microcapsule using the method of the fourth aspect;
(ii) performing the one or more reactions on the biological entity in the microcapsule.

In a sixth aspect the present invention provides a kit for making the microcapsule encapsulating a biological entity according to the method of the fourth, the kit comprising:
(a) an antichaotropic agent and/or a polyhydroxy compound;
(b) a polyampholyte comprising one or more covalently cross-linkable groups; and optionally (c) a microfluidic chip.

In contrast to some of the state-of-the-art methods and techniques the invention described herein does not require sophisticated microfluidic procedures, or DNA oligonucleotide grafting to the polymer mesh. The biological entities, such as cells, are isolated in microcapsules composed of a semi-permeable shell and a polyhydroxy- and/or antichaotropic agent-enriched core. The microcapsules can readily be produced, and are stable, at sizes above 60 μm in diameter. Another important feature of the invention is that, due to the combination of the polyampholytes in the shell and the components of the core, the encapsulated biological entities are preferentially distributed in the core of the capsules, and not at the shell of the capsule, or the core-shell interface. Therefore, the retention of the biological entity is very efficient; e.g. where the biological entity is a mammalian cell, the examples provided herein show that >95% of the cells can be retained inside the capsules during multi-step procedures. When performing nucleic acid assays in capsules the cells and/or cell lysate can be treated multiple times, to not only improve cell lysis efficiency, but also to effectively remove inhibitors that may be present in a cell lysate. Therefore, the entire compartment volume can be replaced with a reaction buffer, which is in sharp contrast to the droplet, nano-well and valve-based microfluidic systems where compartment's reaction composition is adjusted by adding new reagents but not replacing/removing the original ingredients/components.

The microcapsules described herein retain larger biomolecules (e.g. nuclei acid molecules in the range of 100 base pairs and larger) inside, while smaller molecules (such as DNA primers, proteins, lysis and biochemical reagents) can passively move between the core of the microcapsule and the exterior environment of the microcapsule. In addition, the microcapsules are shown herein to be mechanically stable such that they can withstand pipetting, shear forces during flow cytometry analysis, thermocycling, centrifugation at >10,000 rcf, freezing-thawing cycles, and other laboratory procedures, cell lysis, RT-PCR and other multi-step reaction and procedures, yet disintegrate upon the treatment with a desirable stimulus (e.g. adding a chemical or enzyme to destroy the capsule's shell) at a selected step during procedure in order to release the encapsulated material. In particular examples, protease or peptidase enzymes can be used to cleave the peptide bonds in the polyampholyte and release the encapsulated material. This is particularly preferred to avoid damaging the encapsulated material with chemicals that may otherwise be needed to disintegrate the shell.

As a result, the microcapsules described herein have broad applicability in analytical methods, and in particular find use in many biological applications as micro-compartments, e.g. to isolate individual cells, lyse them and then remove lysis reagents as well as inhibitory cell lysate compounds by simply replacing the aqueous buffer in which microcapsules are suspended. Finally, microcapsules containing cell lysate, suspended in a desirable reaction mix are shown herein to enable the nucleic acid analytical procedure of copy DNA synthesis and PCR reaction, demonstrating a simple and well-suited approach for conducting single-cell PCR and single-cell RT-PCR assays on hundreds of thousands of encapsulated cells and molecular species, in a massively parallel fashion.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Nor is the claimed subject matter limited to implementations that solve any or all of the disadvantages noted herein.

Further advantages of the products and methods of the invention will become apparent from the description that follows.

### BRIEF DESCRIPTION OF DRAWINGS

To assist understanding of the present disclosure and to show how embodiments may be put into effect, reference is made by way of example to the accompanying drawings described below. It should be noted that in the schematics provided various aspects are not drawn to scale.

FIG. 1. Schematics of microcapsule generation, biological species isolation, processing and analysis. First a mixture of cells is encapsulated in aqueous two-phase system (water-in-oil) droplets. Other biological species such as mammalian cells, bacterial cells, viruses, proteins or nucleic acid molecules can be encapsulated. The water-in-oil droplets having liquid core and liquid shell are converted to capsules by polymerizing the shell. As described in the main text different approaches can be employed to form polymerized shell. The capsules having polymerized shell can be subjected to chemical, physical or enzymatic treatments (e.g. capsules can be dispersed in RT-PCR mix to initiate nucleic acid amplification). The encapsulated cells are lysed and their RNA is amplified using RT-PCR assay, in which the use of fluorescently labelled DNA primers targeting genes of interest enables differentiation of gene expression based on fluorescent readout. Post RT-PCR capsules can then be analyzed either under fluorescence microscope, on a flow cytometer or fluorescence-activated cell sorting instrument (FACS). 1—cell of type A, 2—cell of type B, 3—cell encapsulation, 4—aqueous phase that will constitute capsules' core, 5—aqueous phase that will constitute capsules' shell, 6—carrier oil, 7—water-in-oil droplet collection, 8—liquid core, 9—liquid shell, 10—polymerization of the shell, 11—aqueous buffer, 12—solidified shell, 13—processing of capsules may involve multi-step procedures by suspending capsules in an aqueous reaction mix to initiate, modify or terminate a reaction, 14—encapsulated cell of type A, 15—encapsulated cells of type B, 16—enzymatic, chemical or physical treatment of capsules, for example the use of PCR, 17—capsules having fluorescence corresponding to the genetic make-up of cell type A, 18—capsules having fluorescence corresponding to the genetic make-up of cell type B, 19—microscopy analysis, 20—flow cytometry or FACS, 21—digital quantification of fluorescent capsules, 22—scatter pot showing different capsule populations

FIG. 2. Schematics and operation of microfluidics system for generation of microcapsules. 1—an inlet for aqueous phase enriched in shell-forming compound; 2—an inlet for aqueous phase enriched in core-forming compound; 3—carrier oil, 4—emulsion collection outlet. (FIG. 2A) Schematics of microfluidics chip and its operation. (FIG. 2B) Example of the microfluidics chip for the generation of capsules. (FIG. 2C) Digital micrographs. Scale bars, 100 μm

FIG. 3. Mammalian cells encapsulated in PEGDA/dextran water-in-oil droplets. The cells encapsulated in water-in-oil droplets composed of the 12% (w/v) PEGDA (MW 2000) and 15% (w/v) Dextran (MW 500K) polymers distributed at the PEGDA/Dextran interface and/or PEGDA phase. Arrows indicate the water-in-oil droplets having a cell. The diameter of the capsule is approximately 75 μm.

FIG. 4. Mammalian cells encapsulated in PEGDA/dextran capsules having a thin shell. The cells (black arrows) escaped compartmentalization during capsule generation. The shell is between 4 and 10 μm thick.

FIG. 5. Mammalian cells encapsulated in PEGDA/dextran capsules having a thick shell. The PEGDA/Dextran capsules with a thick (˜20 μm) shell lost concentricity and the compartmentalized cells (black arrows) tend to escape the compartmentalization through the thinner part of the shell.

FIG. 6. Mammalian cell encapsulation in GMA/dextran capsules. (A) Still photograph of the microfluidics device during GMA/dextran capsule generation. 1—a microchannel with an aqueous phase enriched in shell forming compound (GMA, gelatin methacrylate); 2—a microchannel with an aqueous phase enrich in core-forming compound (dextran); 3—carrier oil, 4—cell. (B) The water-in-oil droplets are converted to (C) microcapsules through gelation and cross-linking. The gelation should be understood as a process during which the liquid-shell is converted into the solidified shell. The cross-linking should be understood as new covalent bond formation between two or more molecules. 5—shell enriched in GMA, 6—core enriched in dextran, 7—temperature-induced gelation, 8—covalent cross-linking, 9—semi-permeable shell composed of polymerized GMA. Scale bars, 100 μm.

FIG. 7. Capsule generation using gelatin with a different degree of methacrylate substitution. Capsules were generated using gelatin/dextran blend where gelatin contained different percentage of methacrylate substitution. For each test 3% (w/v) of gelatin polymer with of a given degree of substitution, and 15% (w/v) dextran (MW˜500 k) were used. (A) gelatin with 0% degree of substitution, (B) GMA with 40% degree of substitution, (C) GMA with 60% degree of substitution, (D) GMA with 80% degree of substitution. Scale bars, 100 μm.

FIG. 8. Capsule generation using GMA with a low-degree of substitution. Capsules were generated using 5% (w/v) GMA with 40% degree of substitution and 15% (w/v) dextran (MW˜500 k). Scale bar, 100 μm.

FIG. 9. Capsule generation using different polymerization approaches. (A) Capsule generation process where cross-linking of the capsule shell was performed during droplet generation step by exposing liquid droplets to photo-illumination. (B) Capsule generation process where cross-linking of the capsule shell was performed by exposing off-chip collected emulsion to photo-illumination. (C) Capsule generation process where at first the capsules' shell was solidified during temperature-induced gelation process, and only then cross-linked by photo-illumination. (D) Capsules, where the shell was polymerized following emulsion collection off-chip, incubation at 4° C. to induce gelation of the shell, dispersed solidified capsules in aqueous buffer only then cross-linked via light-induced polymerization. 1—photo-illumination, 2—capsule collection off-chip, 3—carrier oil, 4—liquid core, 5—chemically cross-linked shell, 6—capsule dispersion in aqueous buffer, 7—water phase, 8—generation of water-in-oil droplets, 9—liquid shell, 10—droplet incubation at low temperature (e.g. 4° C.) to induce the solidification of the shell, 11—solidified shell.

FIG. 10. Capsule generation using temperature-induced and/or light-induced polymerization. Photographs show capsules dispersed in aqueous buffer after polymerization of capsules' shell by temperature-induced gelation and/or light-induced cross-linking. (A) Capsules were generated by cross-linking capsule shell during droplet generation step by exposing droplets to photo-illumination and then dispersed in an aqueous buffer. (B) Capsules, where the shell was polymerized by photo-illumination immediately after emulsion collection off-chip and then dispersed in an aqueous buffer. (C) Capsules, where the shell was polymerized following emulsion collected off-chip, incubation at 4° C. to induce gelation (solidification) of the shell and cross-linking via light-induced polymerization, and then dispersed in an aqueous buffer. (D) Capsules, where the shell was polymerized following emulsion collection off-chip, incubation at 4° C. to induce gelation of the shell, dispersing capsules in an aqueous phase and only then cross-linking via light-induced polymerization. Scale bars, 100 μm.

FIG. 11. Capsule generation using chemical agent-induced polymerization. Photographs show capsules dispersed in an aqueous buffer after polymerization of capsules' shell by chemical agent induced cross-linking and the combination of temperature-induced gelation and chemical agent induced cross-linking. (A) Capsules were generated using GMA/dextran blend where GMA phase was supplemented with 0.3% (w/v) APS, and carrier oil phase was supplemented with 0.4% (v/v) TEMED. The emulsion was collected off-chip and incubated at room temperature for 2 hours to allow sufficient period of time for shell polymerization to occur. Shown are the resulting capsules suspended in an aqueous buffer. (B) The water-in-oil droplets containing GMA/dextran blend were generated and collected off-chip, and incubated at 4° C. for 30 min to induce solidification of the shell. The solidified capsules were then resuspended in an aqueous buffer containing polymerization initiators (0.3% (w/v) APS and 0.4% (v/v) TEMED) and incubated at room temperature for 2 h to induce chemical cross-linking of capsules' shell. Shown are the resulting capsules suspended in an aqueous buffer. Scale bars, 100 μm.

FIG. 12. Capsule production using polyhydroxy compounds. Capsules were generated using a mixture of GMA and polyhydroxy compounds. (A) Capsules composed of GMA and hydroxyethyl-cellulose, solidified and polymerized at ˜4° C. temperature. (B) Capsules composed of GMA and Ficoll PM400, solidified and polymerized at ˜4° C. temperature. Scale bars, 100 μm.

FIG. 13. Capsule production using ion liquids. Capsules were generated using a mixture of GMA and ammonium sulfate. Scale bar, 100 μm.

FIG. 14. Generation of capsules having different diameter. (A) Capsules having a diameter of 35 μm, (B) Capsules having a diameter of 60 μm, (C) Capsules having a diameter of 180 μm, (D) Capsules having a diameter of 24 μm. Scales bars, 100 μm.

FIG. 15. Capsule size control by temperature. (A) Capsules composed of GMA and dextran, were solidified and photo-polymerized at ˜4° C. temperature. (B) Capsules composed of GMA and dextran, were solidified at ˜4° C. temperature and photo-polymerized after 15 minutes incubation at room (˜22° C.) temperature. (C) Capsules composed of GMA and hydroxyethyl-cellulose, were solidified and photo-polymerized at ˜4° C. temperature. (D) Capsules composed of GMA and hydroxyethyl-cellulose, were solidified at ˜4° C. temperature and photo-polymerized after 15 minutes incubation at room (˜22° C.) temperature. (E) Capsules composed of GMA and Ficoll PM400, were solidified and photo-polymerized at ˜4° C. temperature. (F) Capsules composed of GMA and Ficoll PM400, were solidified at ˜4° C. temperature and photo-polymerized after 15 minutes incubation at room (˜22° C.) temperature. Scale bars, 100 μm.

FIG. 16. Increasing the concentration of the shell-forming precursor leads to capsules with thicker shells. The capsules were generated by emulsifying 5% (w/v) GMA with 15% (w/v) dextran solutions followed by physical gelation and light-induced cross-linking of the shell either at ˜4° C. (panel A) or ˜22° C. temperature (panel B). (A) The photograph shows ˜68 μm diameter capsules having 6.5 μm shell and 55 μm core. (B) The photograph shows ˜82 μm diameter capsules having 6 μm shell and 70 μm core. Scale bars, 100 μm.

FIG. 17. Single-cell isolation in capsules. Cell retention comparison in droplets and in capsules. Boxplots (FIG. 17A) and bar plots (FIG. 17B) representing mammalian cell retention in droplets and capsules. Independent samples t-test showed there is no statistically significant difference of cell occupancy in droplets and capsules. (p=0.2281). Cell occupancy in droplets is 8.2±1.6, cell occupancy in capsules is 8.8±1.3. In both FIG. 17A and FIG. 17B values for retention in droplets is shown on left with the values for retention in capsules shown on the right.

FIG. 18. The concept of performing single-cell and nucleic acid analysis using capsules. The individual cells are isolated (compartmentalized) in semi-permeable capsules and are lysed by dispersing capsules in an appropriate lysis buffer. Upon lysis the nucleic acid molecules longer than 200 bp are preferably retained within the capsules, while low molecular weight compounds are removed by washing capsules in washing buffer. Following cell lysis step, the capsules carrying single-cell lysates including nucleic acids are dispersed in RT-PCR reaction mix containing fluorescently labelled oligonucleotides. Once dispersed in aqueous reaction mix, the reaction components quickly diffuse to the capsules enabling multiplex RT-PCR reaction on encapsulated species. After RT-PCR the capsules carrying particular cell type, and/or capsules carrying the transcript(s) of interest, become fluorescent. Using fluorescently labelled primers targeting different transcripts of interest the capsules will acquire a corresponding fluorescence thus enabling differentiation of encapsulated cells based on their transcriptional profile.

FIG. 19. Epifluorescence microscopy analysis of capsules after multiplex RT-PCR. The first (top) row shows multiplex RT-PCR results on capsules carrying a mixture of K562 and HEK293 cells. The second row shows multiplex RT-PCR results on capsules carrying K562 cells. The third row shows multiplex RT-PCR results on capsules carrying HEK293 cells. The fourth row shows multiplex RT-PCR results on capsules carrying a mixture of K562 and HEK293 cells, when reverse transcription enzyme was omitted from reaction mix (no RT). The fifth row shows multiplex RT-PCR results on capsules carrying no template. The first column shows bright field images. The second column shows Alexa Fluor 647 dye fluorescene images corresponding to ACTB positive capsules. The third column shows Alexa Fluor 488 dye fluorescene images corresponding to PTPRC positive capsules. The fourth column shows Alexa Fluor 555 dye fluorescene images corresponding to YAP positive capsules. Fifth column shows merged images demonstrating cell specific markers (PTPRC and YAP) overlapping with ACTB expression. The ACTB positive capsule lacking PTPRC and YAP signal indicate ambient ACTB transcript levels originating from the lysed cells. Scale bars, 100 μm.

FIG. 20. Selected examples of epifluorescence images of capsules after multiplex RT-PCR on fixed cells. The mixture of K562 and HEK293 cells was isolated into capsules at a limiting dilution such that each capsule, on average, would contain no more than one cell. The encapsulated cells were fixed with ethanol, stained with DAPI dye and subjected to multiplex RT-PCR using fluorescently labelled PCR primers targeting cDNA of ACTB, PTPRC and YAP. (A) Digital images of a capsule that contained ambient RNA encoding ACTB. (B) Digital images of a capsule that carries a dead cell. Note, the fluorescence of the cell nucleus in the DAPI, ACTB, PTPRC and YAP channels. (C) Digital images of a capsule that carries HEK293 cell. Note, that cell nucleus exhibits localized fluorescence in the DAPI, ACTB, PTPRC and YAP channels, whereas the ACTB and YAP mRNA amplified during RT-PCR step shows fluorescence signal occupying the core of a capsule. (D) Digital images of a capsule that carries K562 cell. Cell nucleus exhibits localized fluorescence in the DAPI, ACTB, PTPRC and YAP channels, while the ACTB and PTPRC mRNA amplified during RT-PCR step exhibits fluorescence distributed in the core of a capsule.

FIG. 21. Single-cell PCR. The K562 cells, isolated in semi-permeable capsules at a limiting dilution subjected to PCR using fluorescently labelled PCR primers targeting region of interest in the genomic DNA. (A) Digital image of capsules under bright field. (B) Post-PCR capsules stained with SYBR Green I, which binds to PCR amplicons and becomes highly fluorescent. (C) Post-PCR capsules that successfully amplified single-cell genomic DNA region of interest are fluorescent (magenta). (D) Composite image of panels A-C. Scale bars, 100 μm.

FIG. 22. Individual RNA molecule amplification by targeted RT-PCR. The total RNA purified from both K562 and HEK293 cells was mixed, loaded into GMA/dextran capsules and amplified using multiplex RT-PCR with fluorescent primers targeting cDNA encoding ACTB, YAP and PTPRC markers. Scale bar, 100 μm.

FIG. 23. Individual DNA molecule amplification by PCR. The individual DNA molecules were encapsulated in GMA/dextran capsules such that one capsule and amplified by PCR to generate 1.5 kb DNA amplicon. Post-PCR capsules were stained with SYBR Green I dye, which binds to dsDNA and becomes highly fluorescent. Scale bar, 100 μm.

FIG. 24. Capsule analysis using FACS instrument. The FITC-dextran labelled capsules were analyzed on FACS instrument using forward scatter (FSC), side scatter (SSC) and Green fluorescence. (A) Side vs. forward scatter plot of capsule sample. (B) The capsules gated on the forward vs. side scatter plot were re-plotted on the FSC vs. fluorescence scatter plot. (C) The capsules gated on the forward vs. side scatter plot were re-plotted on the SSC vs. fluorescence scatter plot. (D) The digital photograph of FITC-dextran labelled capsules used in the flow cytometry analysis. Scale bar, 100 μm.

FIG. 25. Capsule flow cytometry analysis reveals distinct cell types based on cell-specific marker expression. The plots show three capsule samples containing a mixture of K562 and HEK293 cells (A), K562 cells (B) or HEK293 cells (C). The capsules were first gated based on forward vs. side scatter signal (Capsule gate), the resulting sub-population was then gated on ACTB marker expression (Actin gate) and finally the expression of PTPRC and YAP markers was evaluated on the fluorescence scatter plot (Cell marker fluorescence). Note, that due to the spillover of Alexa Fluor 488 to Alexa Fluor 555 channel and imperfect compensation process, PTPRC-positive population has similar intensity of Alexa Fluor 555 as YAP-positive counts. The ACTB-positive capsules showing low fluorescence signal in Alexa Fluor 488 and Alexa Fluor 555 channels form separate sub-population(s) and indicate the level of ambient RNA molecules and/or dead cells present in the sample. The total amount of capsules analyzed in this experiment was 72497, 78515 and 107798 for K562 & HEK293 (A), K562 (B) and HEK293 (C) samples, respectively.

FIG. 26. Scatter plots of single-cell post-RT-PCR samples. Three different workflows were tested: direct workflow (as described in “Tube 3” processing conditions in the main text), cell freezing (as described in “Tube 1” processing conditions in the main text) and cDNA freezing (as described in “Tube 2” processing conditions in the main text). Selected thresholds that will be used for the downstream analysis: ACTB positive—1.35, PTPRC positive—1.35, YAP positive—1.15.

FIG. 27. Comparison of single-cell RT-PCR results of samples with and without preservation step. Boxplots representing ACTB (left), PTPRC (middle) and YAP (right) positive capsule fluorescence intensities obtained with different sample preparation technique. Three different workflows were tested: direct workflow (as described in “Tube 3” processing conditions in the main text), cell freezing (as described in “Tube 1” processing conditions in the main text) and cDNA freezing (as described in “Tube 2” processing conditions in the main text). One-way ANOVA showed there is no statistically significant difference in mean PTPRC fluorescence between different sample preparations (p=0.352). Statistically significant differences were observed in ACTB fluorescence (p=0.0000443) and YAP fluorescence (p=0.0147). Pairwise comparison using Tukey post hoc test revealed that statistically significant differences in ACTB fluorescence exist between direct workflow and cell freezing (p<0.001) and cell freezing and cDNA freezing (p=0.0033). Statistically significant differences in YAP fluorescence were obtained between direct workflow and cell freezing (p=0.0254) and direct workflow and cDNA freezing (p=0.0382). Number of ACTB-positive capsules used in statistical analysis was 407 (direct workflow—151, cell freezing—124, cDNA freezing—132). Number of PTPRC-positive capsules used in statistical analysis was 147 (direct workflow—54, cell freezing—47, cDNA freezing—46). Number of YAP-positive capsules used in statistical analysis was 155 (direct workflow—51, cell freezing—51, cDNA freezing—53).

FIG. 28. Comparison of RT-PCR results between different sample processing techniques. Boxplots representing ACTB, PTPRC, YAP, ACTB & PTPRC and ACTB & PTPRC amount in post-RT-PCR capsules obtained with distinct processing techniques. Three different workflows were tested: direct workflow (as described in “Tube 3” processing conditions in the main text), cell freezing (as described in “Tube 1” processing conditions in the main text) and cDNA freezing (as described in “Tube 2” processing conditions in the main text). P values were calculated applying nonparametric Kruskal-Wallis test. Note, that all p values are higher than 0.05, which indicates statistically insignificant differences between sample processing techniques.

FIG. 29. Agarose gel showing retention of DNA fragments inside the semi-permeable compartments. The microcapsules subjected to different conditions (see Example 9 for more details). GeneRuler 100 bp Plus DNA ladder (cat no. SM0321, ThermoFisherScientific) was encapsulated in water-in-oil droplets and processed as follow. The encapsulated DNA ladder was released immediately after droplet collection off-chip showing that there is no preferential DNA fragment loss during encapsulation process (well #1). The microcapsules released into aqueous buffer at 4° C. also retain encapsulated DNA fragments and shown now preferential loss (well #2). The microcapsules incubated at room temperature (well #3 and #5) or incubated at 50° C. for 30 min (well #4 and #6) show the same degree of DNA fragment retention. The capsules treated with dextranase (well #5 and #6) did not affect DNA fragment retention. Microcapsules were broken by treating with protease, the released material was combined with DNA loading dye and individual samples were loaded in agarose wells for DNA electrophoresis. Well #M indicates a well having GeneRuler 100 bp Plus DNA ladder.

FIG. 30. Photographs of microcapsules after whole genome amplification of single-cells. The single-cells (K-562 cell line) were isolated in microcapsules, lysed and the genome was amplified using phi29 DNA polymerase. After whole genome amplification (WGA) the microcapsules were stained with fluorescent dyes to evaluate the reaction yields. A) Photographs of single-cell whole genome amplification product stained with SYBR Green I dye. B) Photographs of single-cell whole genome amplification product stained with SYTO-9 dye. After WGA reaction the majority of microcapsules expanded in size due to increased levels of DNA present in the microcapsules. Scale bars, 100 μm.

### DETAILED DESCRIPTION OF THE INVENTION

Throughout the text the terms of ‘comprising’ and ‘containing’ have been used interchangeably and have the same meaning.

The terms “approximately” or “about” are used herein and generally refer to a range of ±30% of the stated value, preferably ±20% of the stated value, more preferably ±10% of the stated value, and even more preferably ±5% of the stated value.

Articles such as “a,” “an,” and “the” include the singular and the plural reference unless the context clearly indicates otherwise. For example, a reference to “a cell” includes one cell and two or more of such cells.

The properties of the microcapsule described below are those at room temperature, i.e. 22° C., unless otherwise specified.

As described above, the present invention relates to microcapsules that can be formed around a biological entity and which can be used to provide a microcompartment in which to perform reactions on the biological entity.

The biological entity may be a cell, a microorganism, a bacteria, a virus, or a biological compound such as a nucleic acid molecule. Preferably the biological entity is a cell, more preferably a eukaryotic cell, such as a mammalian cell or more particularly a human cell.

Where the biological entity is a cell, the cell may be lysed while in the microcapsule such that the biological entity in the microcapsule is a biological compound from the lysed cell. In particular, as will be described further below, the microcapsules described herein have particular utility in the analysis of cells through the compartmentalisation of individual cells in a microcapsule, the lysis of the cell within the microcapsule and the reaction of nucleic acid molecules released from the cell while the nucleic acid molecule is retained within the microcapsule.

The microcapsules described herein comprise a core surrounded by a semi-permeable shell, with the biological entity in the core. The semi-permeable shell permits the passive diffusion of low molecular weight molecules, including reagents such as primers, through the shell, while retaining larger molecular weight molecules, particularly the biological entity requiring compartmentalisation. For example, the semi-permeable shell retains larger biomolecules including nucleic acid molecules longer than 200 nucleotides, preferably longer than 150 nucleotides, and more preferably longer than 100 nucleotides. In another example, the semi-permeable shell allows for diffusion of smaller molecular weight compounds having approximate molecular weight of 120,000±80,000 Da or less through the shell, while retaining larger molecular weight compounds having approximate molecular weight of 300,000±100,000 Da and above. (In particular examples, the microcapsules can contain very large molecular weight compounds including a cell genome, which has a mass of 2.15×10⁹ Da.) In this way, the biological entity within the microcapsule can be contacted with reagents as they diffuse from the external environment of the microcapsule (which may be a reaction buffer in which the microcapsule is suspended) through the semi-permeable shell, into the core. Similarly, reagents and buffer from a previous reaction can be removed from the core by placing the microcapsule in suitable external environment such that the reagents and buffer passively diffuse across the semi-permeable shell into the external environment down a concentration gradient.

The core of the microcapsule may be liquid, semi-liquid or a hydrogel. In particular, the core of the microcapsule may comprise an antichaotropic agent, such as a kosmotrope, and/or a polyhydroxy compound. The kosmotrope anions may be a carbonate, a sulphate, a phosphate or a citrate. In a preferred example the kosmotrope is a kosmotropic salt such as an ammonium sulphate.

The polyhydroxy compound is one selected from a polysaccharide, a carbohydrate, an oligosaccharide, or a sugar. The polyhydroxy compound may be synthetic or naturally occurring. In one example, the polyhydroxy compound is one or more of dextran, alginate, hyaluronic acid, glucan, starch (amylose, amylopectin), agarose, heparin, pectin, cellulose, chitosan, xanthan gum, polyglycerol, and/or the modifications thereof. In particular, the polyhydroxy compound may be one or more of dextran, hydroxyethyl cellulose or a synthetic sucrose polymer, such as Ficoll PM400. Dextran is particularly preferred.

The polyhydroxy compound may have a molecular weight of 300 Da to 5000 kDa. In one example the molecular weight is greater than 10 kDa. In another example the molecular weight is greater than 100 kDa. In a preferred example, the polyhydroxy compound has a molecular weight of 400 to 2000 kDa, more preferably approximately 500 kDa.

The semi-permeable shell of the microcapsule comprises a gel formed from a polyampholyte. The term ‘polyampholyte’ refers to a polyelectrolyte that bears both cationic and anionic groups, or corresponding ionizable groups, and where the ‘polyelectrolytes’ are polymers whose repeating units bear an electrolyte group. It should be understood that term ‘polyampholyte’ and ‘ampholytic polymer’ are synonyms as defined by IUPAC. In a preferred embodiment the polyampholytes in the gel are polymers comprising peptide bonds, wherein the polymers are covalently cross-linked into an elastic gel. Specifically, individual linear polymer strands are cross-linked to each other to create a polymer mesh, i.e. the cross-links comprise intermolecular cross-links.

In particular, the polymer may comprise a protein, peptides, oligopeptides or polypeptides, or any combination thereof. Accordingly, the polymer may be described as “proteinaceous”. In particular, in some embodiments of this invention the polyampholyte is a protein, polypeptides or oligopeptides with a primary amino acid sequence comprises at least 10% disorder promoting amino acids, and preferably at least 30%. Disorder promoting amino acids include proline, glycine, glutamic acid/glutamate, serine, lysine, alanine, arginine, and glutamine. Without wishing to be bound by theory it is considered that such disorder promoting amino acids also promote liquid-liquid phase separation in the droplet during formation of the microcapsule (which is discussed further below). Furthermore, the proteinaceous polyampholytes that show liquid-liquid phase separation properties are often characterized by long segments of low diversity amino acids. These segments are often repetitive and are enriched in glycine (G), glutamine (Q), asparagine (N), serine (S), arginine (R), lysine (K), aspartate (D), glutamate (E) or aromatic amino acids such as phenylalanine (F) and tyrosine (Y) amino acids. These segments often encompass multiple short motifs such as YG/S-, FG-, RG-, GY-, KSPEA-, SY- and Q/N-rich regions, or regions of alternating charges [21].

In some examples, the polymers are “thermo-responsive” polymers. “Thermo responsive” polymers are those that are capable of undergoing a transformation when subjected to a change in temperature. In the context of the present invention the “thermo responsive” polymers are capable of forming a gel when subjected to a change in temperature, for example when cooled, below sol-gel transition temperature. The gel that is formed is a mesh or 3-dimensional network of polymer strands, which maintain a solid structure due to physical cross-linking of individual polymer strands.

The structure of the semi-permeable shell is further stabilized by covalent cross-linking between the polymer strands. In particular, the polymers from which the semi-permeable shell is formed may comprise one or more covalently cross-linkable groups which can be used to form the covalent cross-linking. In some embodiments the polyampholyte is modified with one or more chemical groups which can be used for the covalent cross-linking. Examples of suitable chemical groups are acrydite, acrylate methacryloyl, acrylamide, methacrylamide, bisacrylamide, methacrylate, methacrylic acid, acrylic acid, polyacrylic acid, methacrylic anhydride, acryloyl, vinyl, vinylsulfone, vinylpyrrolidone, thiol, disulphide, cystamine, carboxyl, amine, imine, azide, triazole, tetrazine, azidophenylalanine, alkynyl, alkenyl, alkyne, diisocyanate, hydroxypropionic acid, hydroxy phenol, azobenzene, methylcyclopropene, trans-cyclooctene (TCO), norbornene, diarylcyclooctyne (DBCO), or cyclooctanyl moieties and/or reagents.

The degree of substitution of these chemical groups on the polymer can be varied to achieve the desired microcapsule stability, which will depend on the nature of the reactions that are to be performed on the biological entity in the microcapsule and/or the processes to which the microcapsule is to be subjected.

In one example, the polymers are extra-cellular matrix proteins, or peptides, oligopeptides, polypeptides or proteins derived from extra-cellular matrix proteins. The extracellular matrix proteins may be collagen, laminin, elastin, laminin, proteoglycans, or glycosaminoglycans.

The polyampholyte may be derivatives that may be hydrolyzed forms of proteins. The preferred polymer is hydrolyzed collagen, particularly gelatin.

The polyampholyte may be derivatives elastin-like polypeptides, fibrin, silk fibrion, glycinin, gluten, casein, or hydrolyzed forms thereof.

As noted above, the gelatin may be modified with one or more chemical groups. Preferred examples are gelatin methacryloyl, gelatin methacrylamide gelatin acrylamide, and gelatin methacrylate, while gelatin methacrylate is particularly preferred. The gelatin may have a degree of substitution of 10 to 90%, preferably 40 to 90%, and more preferably 60 to 80%.

The shell and core of the microcapsule are approximately concentric, and the microcapsule may be about 1 μm to about 1000 μm in diameter, about 20 μm to about 200 μm in diameter, and preferably about 60 to about 150 μm in diameter. In particular, the size of the microcapsules can be selected depending on the size of the biological entity it is to be used to encapsulate.

The shell of the microcapsule may be in the range of 0.1 to 100 μm and more preferably in the range of 1 to 20 μm in thickness, preferably in the range of 2 to 10 μm.

Microcapsule dimensions can be measured from images taken with a microscope.

In particular, the microcapsules of the present invention can be produced around a biological entity by a method comprising:
(a) forming a water-in-oil droplet comprising a first solute, a second solute and the biological entity, wherein the first solute is a polyampholyte and the second solute is an antichaotropic agent and/or a polyhydroxy compound, wherein the polyampholyte comprises one or more covalently cross-linkable groups;
(b) allowing aqueous phase separation inside the water-in-oil droplet into a shell phase enriched in the first solute and a core phase enriched in the second solute, and gelation and/or precipitation in the shell phase to form an intermediate microcapsule;
(c) forming intermolecular covalent cross-links with the one or more covalently cross-linkable groups to form the microcapsule comprising a semi-permeable shell of covalently cross-linked polyampholyte and a core, wherein the biological entity is in the core.

Water-in-oil droplet generation methods are well described and are known to the skilled person in the art [22-29] including Torii et al., JP Pub. No. 2004/083802; Link et al., WO 2004/091763; Weitz et al., U.S. Pub. No. 2009/0012187; Bibette et al., WO 2010/063937; Weitz et al. U.S. Pub. No. 2012/0211084; Weitz et al., U.S. Pub. No. 2013/0064862. Droplets may be generated in so called dripping mode or in so called jetting mode. In a preferred case scenario, an emulsion comprising water-in-oil droplets may be generated using a microfluidic system. In particular, the microfluidics system may utilise a droplet generation device manufactured by soft-lithography to generate the aqueous droplet in a carrier oil. However, it would be understood by a person skilled in the art that microfluidics devices can be produced from different materials (e.g. plastic) or can be replaced with analogous systems to generate aqueous droplets in a carrier oil. For example, glass capillary devices could be used for this purpose to isolate individual cells in droplets [30]. The method may be also performed using extrusion, shaking, agitation, micro-sieve and capillary assemblies or other droplet generation systems.

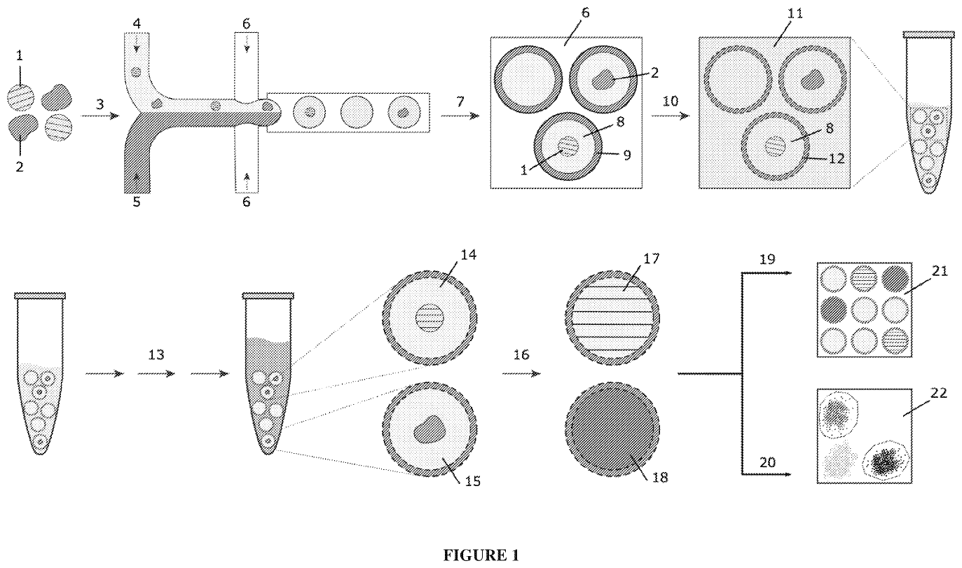

A schematic of an example of the method is shown in FIG. 1.

In (a) and (b) a biological entity or a plurality of biological entities, is/are partitioned into one or a plurality of liquid droplet(s). In particular, (a) may be performed by mixing a first solution comprising the polyampholyte as a first solute with a second solution comprising the polyhydroxy compound and/or the antichaotropic agent as a second solute, and simultaneously or separately combining the first and second solution with a carrier oil. The carrier oil should comprise a surfactant to prevent coalescence of the droplets [31]. The biological entities may be included in one of the solutions, preferably in the second solution, or may be comprised in a third solution that is combined with the first and second solutions.

Suitable carrier oils and surfactants for the production of water-in-oil droplets are known in the art. In a particular embodiment, the carrier oil used to generate droplets is a fluorinated oil and comprises a surfactant, a PFPE-PEG-PFPE (perfluoropolyether-polyethylene glycol-perfluoropolyether) tri-block copolymer, or PEG-PFPE (polyethylene glycol-perfluoropolyether) di-block copolymer. Said surfactant(s) being present in the carrier oil at a concentration ranging from 0.05% to 5% (w/w), preferably ranging from 0.1% to 1% (w/w), more preferably ranging from 1% to 3% (w/w). The method of the present invention is not limited by the type of surfactant or carrier oil used. One of ordinary skill in the art will be able to select the appropriate surfactant and carrier oil.

In an embodiment, the carrier oil is selected from the group consisting of fluorinated oil such as FC40 oil (3M®), FC43 (3M®), FC77 oil (3M®), FC72 (3M®), FC84 (3M®), FC70 (3M®), HFE-7500 (3M®), HFE-7100 (3M®), perfluorohexane, perfluorooctane, perfluorodecane, Galden-HT135 oil (Solvay Solexis), Galden-HT170 oil (Solvay Solexis), Galden-HT110 oil (Solvay Solexis), Galden-HT90 oil (Solvay Solexis), Galden-HT70 oil (Solvay Solexis), Galden PFPE liquids, Galden® SV Fluids or H-Galden® ZV Fluids; and hydrocarbon oils such as Mineral oils, Light mineral oil, Adepsine oil, Albolene, Cable oil, Baby Oil, Drakeol, Electrical Insulating Oil, Heat-treating oil, Hydraulic oil, Lignite oil, Liquid paraffin, Mineral Seal Oil, Paraffin oil, Petroleum, Technical oil, White oil, Silicone oils or Vegetable oils. In a particular embodiment, the carrier oil is a fluorinated oil. In a more particular embodiment, the carrier oil is HFE-7500 oil.

The polymers, the polyhydroxy compound and/or the antichaotropic agent may be as defined above in relation to the microcapsule. The amounts of the polymer in the first solution and the amount of the polyhydroxy compound and/or antichaotropic agent in the second solution may be varied in order to vary the size and stability of the microcapsules. In one example the first solution comprises 0.1 to 20% (w/v) of the polymer, preferably 1 to 15% (w/v). Where the polymer is gelatin, or gelatin derivative, in the first solution may preferably comprise about 3 to 4% (w/v) of the polymer. Alternatively, or in addition, the second solution comprises 0.1 to 40% (w/v) of the polyhydroxy compound. Preferably, where the polyhydroxy compound is dextran the second solution comprises 3 to 30% (w/v) dextran. Where the polyhydroxy compound is Ficoll, the second solution may comprise 3 to 30% (w/v) Ficoll.

In (b) the method may comprises transforming the liquid shell phase into a thermoreversible gel by changing the temperature of the water-in-oil droplet so as to induce physical cross-linking of the thermo-responsive polymer strands.

For example, in this step the water-in-oil droplet may be cooled below 40° C., below room temperature (around 22° C.), below 10° C., and preferably to about 4° C., in order to induce gelation of the polyampholyte into a gel. In particular, without wishing to be bound by theory, the inventors consider that performing a cooling step prior to the covalent cross-linking step, enables the polymers in the gel to have a more ordered structure which ultimately can result in a more stable microparticle and open broader possibilities for performing a cross-linking reaction. Moreover, the gelled (solidified) shell may protect the biological entity from the reagents used in the subsequence chemical cross-linking step.

The water-in-oil droplet may be incubated at the low temperature for a period of 1 minute to 1 hour, preferably between 5 to 45 minutes.

As noted above, the polyampholyte comprises one or more covalently cross-linkable groups for the covalent cross-linking in (c). In particular the polymer may be modified with these groups. In one example the polymer has a degree of substitution of 20 to 90%, preferably 40 to 90%, more preferably 60 to 80%. It is thought that degrees of substitution lead to more covalent cross-linking and produce a more stable microparticle.

Step (c) may comprise exposing the intermediate microcapsule to a chemical agent, light or heat, or any combination thereof, to covalently cross-link the polymer. In particular, (c) may comprise activating the chemical group by exposing the liquid shell phase or the gel to an initiator, such as chemical-initiator (e.g., tetramethylethylenediamine, ammonium persulfate), photo-initiator (e.g., lithium phenyl-2,4,6-trimethylbenzoylphosphinate), thermal initiator (e.g., heat), radiative-initiator (e.g., visible or UV light), or any combination thereof. Preferably (c) comprises covalently cross-linking by photo-polymerisation.

Differently sized microcapsules can be produced by varying the volume of the solutions used in the step of forming the water-in-oil droplet, for example, by varying the flow rates or cross-sections of the microfluidic channels if the microcapsule is being produced in a microfluidic device, and/or by bringing the microcapsules to room temperature prior to covalent cross-linking to induce capsule swelling and expansion.

The compartmentalisation of the biological entity in the microcapsule described herein permits one or more reaction to be performed on the biological entity while it is retained in the microcapsule. Due to the semi-permeability of the shell, the microcapsules can be suspended in an aqueous reaction mix comprising one or more components for performing a reaction. The one or more components then diffuse across the semi-permeable shell into the core, and contact the biological entity, thus bringing about the reaction. The one or more components can be one or more reagents, one or more proteins, one or more enzymes and one or more substrates. In addition, the microcapsule may be subjected to external conditions, such as incubation at a specific temperature, as part of the reaction conditions.

In particular, the one or more reactions may be selected from labelling the biological entity, analysing the biological entity, releasing a component from the biological entity, and optionally may further comprise labelling a component released from the biological entity, and/or analysing a component released from the biological entity.

In some preferred examples, the one or more reactions may be cell lysis followed by nucleic acid analysis. Nucleic acid purification and/or chemical and/or enzymatic treatment may follow cell lysis in order to remove the inhibitory effects of a cell lysate. In particularly preferred embodiments the nucleic acid analysis comprises reverse transcriptase (RT) to produce cDNA followed by an enzymatic amplification and/or labelling of the DNA, in order to obtain genetic make-up information or genome-encoded information about the encapsulated cells.

Specifically, the nucleic acid amplification by enzymes, such as DNA polymerase driven polymerase chain reaction, may be used to generate fluorescently labelled DNA. The presence of fluorescently labelled DNA in a microcapsule may then be detected by digitally recording the fluorescence, or by flow cytometry, or by sorting the microcapsules using a FACS instrument, or other light recording device or instrument. In a typical case scenario encapsulated cells are lysed and their RNA is amplified using RT-PCR assay, in which the use of fluorescently labelled DNA primers targeting genes of interest enables differentiation of gene expression based on fluorescent readout. Post RT-PCR capsules can then be analyzed either under fluorescence microscope, or on a flow cytometer.

In addition, the microcapsule can be readily broken to release the products of the one or more reactions performed on the biological entity. Breaking of the capsule can be performed with one or more proteases to cleave the peptide bonds, thus digest the polymer of the semi-permeable shell.

Also provided by the present invention is a kit for producing the microcapsule described herein, the kit comprising an antichaotropic agent and/or a polyhydroxy compound, the polyhydroxy compound being selected from a polysaccharide, a carbohydrate, an oligosaccharide, or a sugar. The kit further comprises a polyampholyte comprising peptide bonds and one or more covalently cross-linkable groups. The antichaotropic agent, the polyhydroxy compound and the polyampholyte may be as described above in relation to the microcapsule. The kit may optionally comprise a microfluidic chip and/or microfluidics consumables (e.g., tubing, needles, etc.). In particular, the microfluidic chip comprises microchannels configured to form a water-in-oil droplet from a first solution comprising the polyhydroxy compound and/or the antichaotropic agent, a second solution comprising the polyampholyte, and a carrier oil with or without surfactant. The kit may further comprise instructions for performance of the methods described herein.

The examples which follow are to be understood as illustrative examples of embodiments of the invention. Further embodiments and examples are envisaged. Any feature described in relation to any one example or embodiment may also be used in combination with one or more features of any other of the examples or embodiments, or any combination of any other of the examples or embodiments, except where the circumstances dictate otherwise.

Furthermore, equivalents and modifications not described herein may also be employed within the scope of the invention, which is defined in the claims.

### EXAMPLES

### Example 1: Development of Semi-Permeable Capsules for Efficient Mammalian Cell Encapsulation and Retention

We have recently reported a method for production of the semi-permeable capsules, composed of polyethylene glycol diacrylate (PEGDA) and dextran [14]. In the published work we showed a few examples of semi-permeable capsule use for genotypic and phenotypic analysis of individual bacterial cells by performing multi-step workflows on hundreds and thousands of cells simultaneously. However, capsules approaching 50-60 μm size were increasingly challenging to produce and they tended to lose the concentricity.

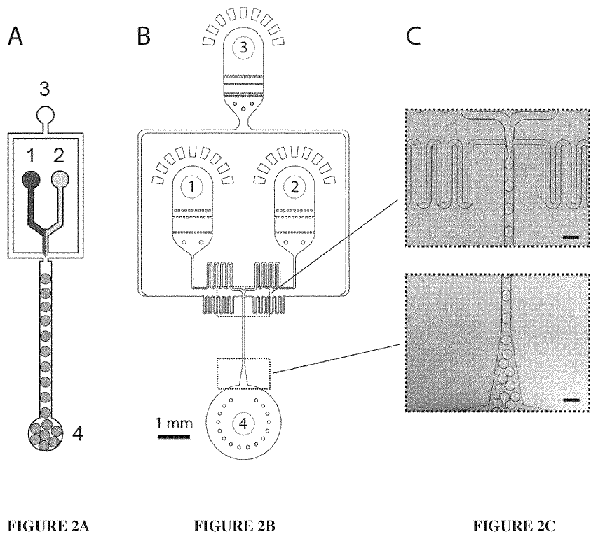

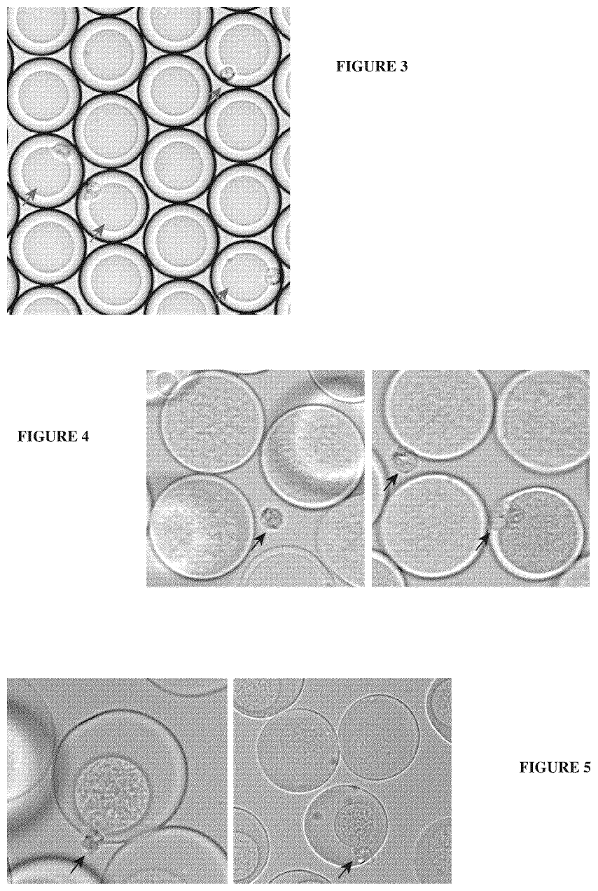

Following the protocol reported previously [14] we attempted to isolate the mammalian cells into a variety of capsules ranging from ˜70 to 90 μm in size. The capsules were composed of PEGDA/dextran blend and were generated using a microfluidic device depicted in FIG. 2. We found that encapsulated mammalian cells preferentially moved to the PEGDA phase and/or arranged themselves at the PEGDA/Dextran interphase (FIG. 3). When mammalian cells were encapsulated in the PEGDA/Dextran capsules, those shell was in the range of 4-10 μm thick, the cells tend to escape the compartmentalization (FIG. 4), resulting in a significant number of capsules void of cells. To improve the cell retention, the capsule shell was increased close to, or larger than, the size of the cell (˜20 μm). However, while increasing the shell thickness visibly reduced the number of prematurely released cells, yet thicker shell did not prevent cells from entering the PEGDA phase (FIG. 5). More importantly, the capsules with a thicker shell lost the concentricity, which led to capsule with uneven shell thickness, which in turn caused cell escape through the thinner shell part (FIG. 5). Others in the field have also noticed that mammalian cells tend to move to PEG-rich phase when working with liquid water-in-oil droplets composed of PEG/dextran blend [32]. Therefore, our results as well as literature reports indicate that previously reported polymer composition of the capsule's core/shell, while suitable for isolation of small bacterial cells [14], is less suitable for efficient retention of mammalian cells. Importantly, the release of encapsulated species in the PEGDA/dextran capsules relies on treatment with harsh chemicals (e.g. 0.1M NaOH solution), which in some circumstances may be damaging to chemically labile species, and cells.

Poor retention of larger cells in PEGDA/Dextran capsules prompted us to search for a novel composition of polymer blends that would meet following requirements: 1) the polymer blend should enable generation of semi-permeable capsules larger than 30 μm in size and retain high core-shell concentricity, 2) the resulting capsules should be compatible with biochemical and biological reactions, 3) the capsules should retain >90% of encapsulated mammalian cells, 4) the capsules should allow passive exchange of assay reagents such as salts, oligonucleotides, yet retain large molecular weight compounds such as messenger RNA (mRNA) or genomic DNA, and 5) the capsules should decompose upon a mild chemical or enzymatic treatment in order to release the encapsulated cells and/or material. We postulated that reducing the amount of PEGDA component in the shell, or replacing it entirely, we will alter the hydrophobic/hydrophilic properties of the shell and will prevent cells from entering the shell, and thus from reaching out the exterior environment. We reveal here that the methods and microcapsules of the invention fulfill the above requirements and can produce highly concentric capsules amenable to single-cell and nucleic acid analysis methods.

To produce gel capsules composed of proteinaceous shell we exemplify the use of gelatin derivative, a thermo-responsive protein, that solidifies at lower temperatures [33]. The rheological properties of the gelatin-based gels can be controlled by the degree of substitution, polymer concentration, initiator concentration, and UV irradiation conditions [33]. Note, that other proteins and oligopeptides, including but not limited to collagen, elastin, fibrin and silk fibroin may be compatible with our approach as well [34].

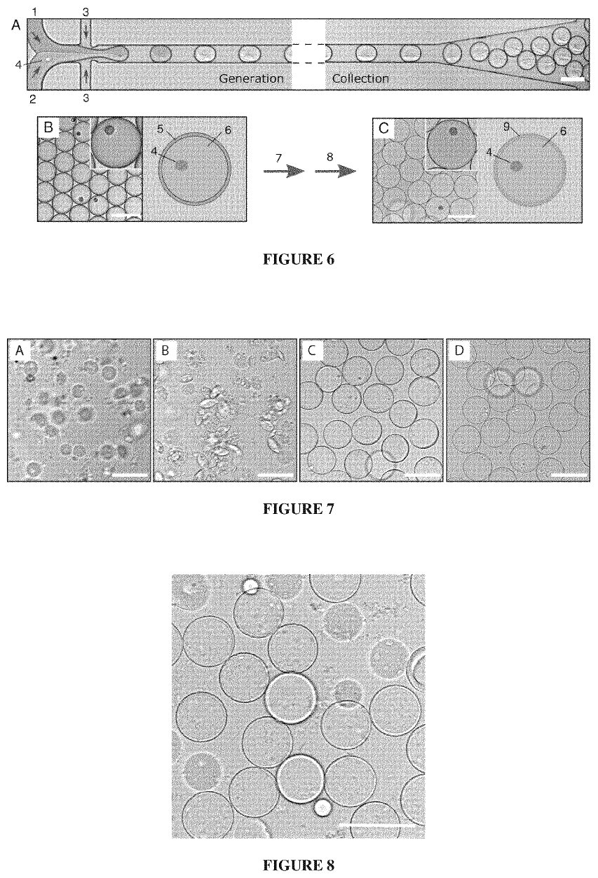

We first generated water-in-oil droplets on a 40-μm deep co-flow microfluidics device using 3% (w/v) gelatin methacrylate (GMA) and 15% (w/v) dextran (MW˜500 k) solutions (FIG. 6a). In the experiments where the cell encapsulation was performed, the cells were suspended in dextran solution accordingly. Typical flow-rates used were: for gelatin methacrylate solution—250 μl/h; for dextran solution (with or without cells)—100 μl/h and for the carrier oil—700 μl/h. After emulsification step, the resulting emulsion droplets contained a well-centered liquid core enriched in dextran and a liquid shell enriched in GMA (FIG. 6b). The droplets were subjected to two-step polymerization process (FIG. 6c). At first, the droplets were incubated at selected temperature that induce the sol-gel transition and solidification of the gelatin shell. Next, the resulting solidified capsules were recovered from the emulsion by breaking the emulsion, re-suspended in an aqueous buffer containing photo-initiator and photo-illuminated to induce chemical cross-linking of methacrylate. More specifically, after producing water-in-oil droplets, the emulsion was transferred onto ice (˜4° C.) and incubated for ˜30 minutes to induce temperature responsive physical gelation of GMA phase. Continuing procedure on ice, capsules were recovered from the emulsion using commercial emulsion breaker (Droplet Genomics, DG-EB-1) and released into 1×PBS buffer supplemented with 0.1% (w/v) Pluronic F-68. Capsule suspension was transferred by pipetting into a new 1.5 ml tube, supplemented with 0.1% (w/v) lithium phenyl-2,4,6-trimethylbenzoylphosphinate (LAP) (Sigma-Aldrich, 900889-1G) and photo-polymerized under exposure to 405 nm light emitting diode (LED) device (Droplet Genomics, DG-BR-405) for 20 seconds. Following the aforementioned two-step polymerization procedure, we could reproducibly generate the capsules with a clear, well-centered core enriched in liquid dextran, and a solidified gel shell composed of covalently cross-linked gelatin (FIG. 6c).

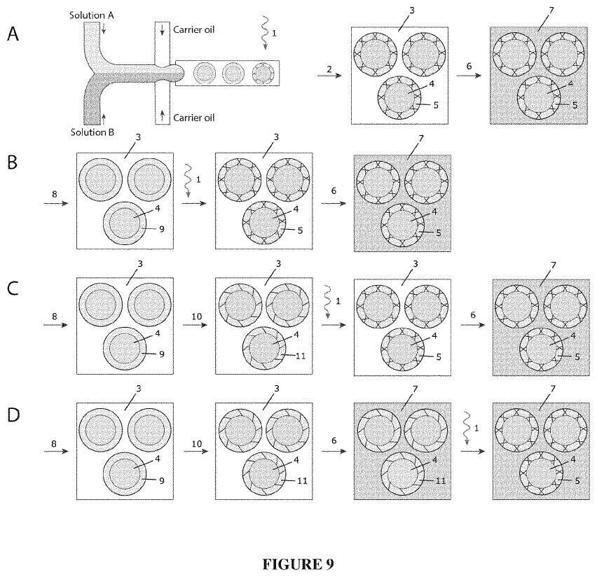

Gelatin methacrylate (GMA) will form a gel of different Young modules depending on the degree of substitution [33]. We investigated what degree of substitution in GMA is required to achieve stable and concentric capsules. We tested capsule production using GMA with 0, 40, 60 and 80% degrees of substitution. For each test 3% (w/v) GMA with a given degree of substitution, and 15% (w/v) dextran (MW˜500 k) was used. Following the aforementioned procedure, we generated capsules and evaluated their quality under bright field microscope. Results presented in FIG. 7 show that GMA with 60 and 80% substitution ensured stable capsule generation, while GMA with 0-40% substitution failed to generate stable capsules. Increasing the GMA amount from 3% to 5% (w/v), while keeping dextran at 15% (w/v), the capsules could be generated with GMA having 40% degree of substitution (FIG. 8). Therefore, production of stable capsules depends on both the GMA substitution degree, which preferably should be at or above 40% and on the total amount of GMA used in the mix, which preferably should be at or above 3% (w/v). Stable capsule production also depends on GMA concentration, temperature, aqueous buffers.

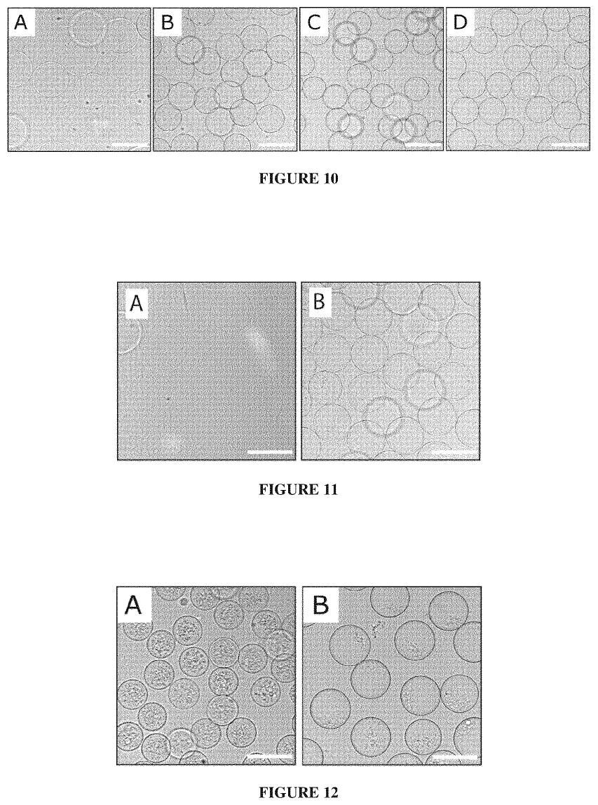

Indeed, it should be understood that there are multiple paths for producing capsules composed of proteinaceous shell and liquid core as well as capsules composed of proteinaceous shell and semi-liquid core. Some of these approaches, but not limited to, have been verified experimentally and are schematically shown in FIG. 9. In a typical scenario, the water-in-oil droplets are incubated at selected temperature that is needed to induce sol-gel transition of the shell phase (e.g. at 4° C. for 30 min). The resulting solidified capsules are then released into aqueous buffer (preferably at temperature that is below the shell melting temperature) for a desired period of time followed by chemical cross-linking of the gel shell. Different variations of this methodology are possible and some are described below:
In one example (FIG. 9A), the shell of capsules are cross-linked during droplet generation step, but only if liquid shell and liquid core has phase separated, by exposing droplets to photo-illumination. The phase separation may be a fast process and happen within a minute, or longer time scales, after water-in-oil droplet is generated. Next, the capsules are dispersed in aqueous buffer (e.g. 1×PBS buffer). The resulting capsules imaged under bright field microscope are shown in FIG. 10A.
In another example (FIG. 9B), the shell of capsules are covalently cross-linked after the emulsion collection off-chip, by photo-illuminating the collected water-in-droplets. Once polymerized, the capsules are dispersed in aqueous buffer and imaged under bright field microscope as shown in FIG. 10B.
In yet another example (FIG. 9C), the shell of capsules are polymerized in a two-step process: following emulsion collection off-chip, the liquid shell of droplets are solidified by incubating droplets at a temperature below sol-gel transition point (e.g. 4° C.) and then further cross-linked by photo-polymerization. Next, the capsules having a covalently cross-linked shell are dispersed in aqueous buffer and imaged under bright field microscope as shown in FIG. 10C.
In yet another example (FIG. 9D), the shell of capsules are polymerized in a two-step process where at first the emulsion droplets are incubated at a temperature below sol-gel transition point (e.g. 4° C.) to induce gelation of the shell, then resulting capsules are dispersed in aqueous phase and only then the shell is cross-linked by photo-polymerization.

The resulting capsules are shown in FIG. 10D.

It should be understood that gel capsules having solidified shell and liquid or semi-liquid core can be generated using different means of polymerization. In a separate example, the shell of capsules can be cross-linked using chemical agent(s). The capsules shown in FIG. 11A were generated by supplementing the GMA phase with 0.3% (w/v) Ammonium Persulfate (APS), while carrier oil was supplemented with 0.4% (w/v) Tetramethylethylenediamine (TEMED). The emulsion was collected off-chip and incubated at room temperature for 2 hours to allow sufficient period of time for shell polymerization to occur. The resulting capsules were suspended in aqueous buffer and evaluated microscopically as shown in FIG. 11A.

In yet another example (FIG. 11B), the shell of capsules are polymerized using a combination of physical and chemical means. The water-in-oil droplets composed of GMA/dextran phases are collected off-chip and incubated at 4° C. for 30 min to induce physical gelation of the shell. The solidified capsules were then resuspended in aqueous buffer (lx DPBS, 0.1% (w/v) F-68) containing 0.3% (w/v) APS, 0.4% (w/v) TEMED, and incubated at room temperature for 2 h to induce chemical cross-linking of capsules' shell. The resulting capsules are shown in FIG. 11B.

It should be understood that the core of capsules is not constrained to the use of dextran. Other polyhydroxy compounds (e.g. carbohydrates, natural and synthetic polymers rich in hydroxy groups, sugars, oligosaccharides, polysaccharides) can be used instead of dextran, or different ratios of polyhydroxy compounds can be mixed at different ratios.

For example, FIG. 12 shows capsules where the dextran phase was entirely replaced with hydroxyethyl-cellulose (FIG. 12A) or Ficoll PM400 (FIG. 12B). In one example generation of capsules is achieved by replacing the dextran phase with 30% (w/v) Ficoll PM400 (Sigma-Aldrich, GE17-0300-10) solution in 1×PBS buffer. In a second example, generation of capsules is achieved by replacing the dextran phase with 3% (w/v) hydroxyethyl-cellulose (Sigma-Aldrich, 09368-100G) solution in 1×PBS buffer. In both examples the aqueous phase forming the shell contained 3% (w/v) GMA in 1×PBS buffer. Also, in both examples the capsules were generated using a microfluidics chip having 40 μm deep channels and flow rates for GMA solution were 250 μl/h, for Ficoll PM400 or hydroxyethyl-cellulose—100 μl/h and for carrier oil—700 μl/h. Emulsifications were performed at room temperature and resulting droplets were collected off-chip into 1.5 ml tube. The collected droplets were incubated at 4° C. for 30 minutes to induce gelatin solidification and thereby the capsule's shell formation. The capsules were then resuspended in an ice-cold 1×PBS buffer supplemented with 0.1% Pluronic F-68 and 0.1% (w/v) LAP and photo-polymerized under exposure to 405 nm LED device for 20 seconds. The resulting capsules were inspected under the bright field microscope and are shown in FIG. 12A and FIG. 12B.

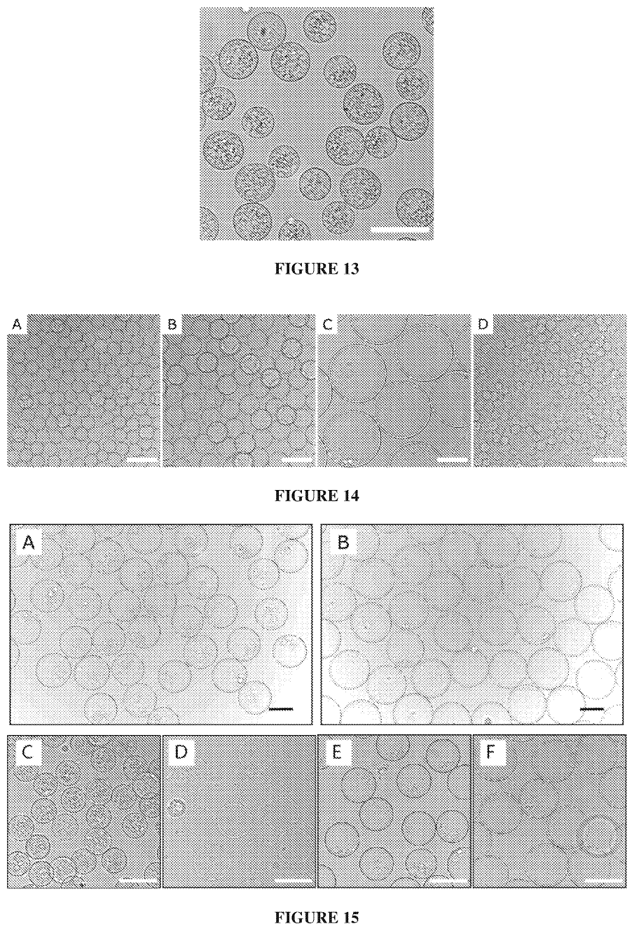

Having shown capsule production using different polyhydroxy compounds, in the following we reveal that capsules can be also generated where the core of the capsules is composed of an antichaotropic agent. To demonstrate such a possibility, we generated capsules where the core forming phase was ionic liquid based on ammonium sulfate. Capsules were generated using a microfluidics chip having 40 μm deep channels and the flow rate for 3% (w/v) GMA solution at 175 μl/h, for 1M ammonium sulfate at 175 μl/h and for carrier oil at 700 μl/h. Emulsification was performed at room temperature and resulting droplets were collected off-chip into 1.5 ml tube. The collected droplets were then incubated at 4° C. for 30 minutes to induce gelatin solidification and thereby the capsule's shell formation. Continuing procedures on ice, the capsules were recovered from the emulsion and resuspended in an ice-cold 1×PBS buffer supplemented with 0.1% (w/v) Pluronic F-68 and 0.1% (w/v) LAP and photo-polymerized under exposure to 405 nm LED device (Droplet Genomics, DG-BR-405) for 20 seconds. The resulting capsules were inspected under the bright field microscope and are shown in FIG. 13.

Although in the examples above preferably only the shell of capsules is solidified while keeping the core of the capsules in a liquid form, it should be understood for the experienced in the field that capsule core can be also polymerized into a desirable strength gel mesh by adding a cross-linking agent soluble in the core phase (or alternatively soluble in both core/shell forming phases). In such case the capsules will contain solidified shell and solidified core of different stiffness. One such example has been revealed by our previous invention (US20200400538A1), where the cross-linking agent, PEGDA, distributed in both phases yet with a higher fraction at the shell phase.

When using a microfluidics device having channels of different depth ranging from 20 to 80 μm deep, the size of capsules could be tuned between 35 and 200 μm by simply changing the flow rates of the system, without significantly affecting their concentricity, thus offering flexibility in size for diverse assay. More specifically, using microfluidics device 20 μm deep the size of capsules was in the range of 35 to 45 μm (FIG. 14A). Using microfluidics device 40 μm deep the size of capsules could be tuned between 60 and 85 μm (FIG. 14B). Using microfluidics device 80 μm deep the size of capsules was in the range of 150 to 200 μm in diameter (FIG. 14C). It should be possible to generate even smaller or larger capsules by employing a microfluidic system having a smaller or larger cross-section channels, or having a smaller/larger size nozzle, respectively. Alternatively, the smaller size capsules can be generated by employing a geometrically mediated breakup of droplets [35]. For example, capsules shown in FIG. 14D were generated using a geometrically mediated breakup, which resulted in 24 μm diameter capsules.

As explained below capsule size can be controlled not only by the flow rates or the cross-section of the microfluidic channels, but also by the temperature. To prove the effect of temperature on capsules size the GMA/dextran capsules were generated using a microfluidics chip having 80 μm deep channels. The flow rates for GMA solution were 200 μl/h, for dextran—50 μl/h and for carrier oil—500 μl/h. Emulsion was collected off-chip into 1.5 ml tube at room temperature and placed at 4° C. for 30 minutes to induce gelatin solidification. Next, emulsion was divided into two fractions and processed separately at different temperatures.
The capsules in the first fraction were recovered from the emulsion, resuspended in an ice-cold 1×PBS buffer supplemented with 0.1% (w/v) Pluronic F-68 and 0.1% (w/v) LAP, and photo-polymerized under 405 nm wavelength for 20 seconds. The resulting capsules are shown in FIG. 15A.
The capsules in the second fraction were recovered from the emulsion, resuspended in an ice-cold 1×PBS buffer supplemented with 0.1% (w/v) Pluronic F-68 and 0.1% (w/v) LAP, and incubated at room temperature (˜22° C.) for 15 min to allow capsule swelling to occur. Following incubation, the capsules were photo-polymerized under 405 nm wavelength for 20 seconds. The resulting capsules are shown in FIG. 15B.

In yet another example the effect of temperature on capsule size is revealed by producing capsules with a core composed of polyhydroxy compounds other than dextran. The capsules of choice were composed of GMA/hydroxyethyl-cellulose or GMA/Ficoll PM400 blend. In one example, the first aqueous phase contained 30% (w/v) Ficoll PM400 solution in 1×PBS buffer and the second aqueous phase contained 3% (w/v) GMA in 1×PBS. In another example, the first aqueous phase contained 3% (w/v) hydroxyethyl-cellulose solution in 1×PBS and the second aqueous phase contained 3% (w/v) GMA in 1×PBS buffer. In both examples the capsules were generated using a microfluidics chip having 40 μm deep channels and flow rates for GMA phase at 250 μl/h, for Ficoll PM400 or hydroxyethyl-cellulose—100 μl/h and for carrier oil—700 μl/h. Emulsifications were performed at room temperature. The collected droplets were incubated at 4° C. for 30 minutes to induce gelatin solidification and thereby the capsule's shell formation. Next, emulsion was divided into two fractions. The emulsion in the first fraction was further processed on ice, whereas the emulsion in the second fraction was processed at room temperature following incubation on ice.
The capsules in the first fraction were dispersed in an ice-cold 1×PBS buffer supplemented with 0.1% (w/v) Pluronic F-68 and 0.1% LAP, and photo-polymerized under 405 nm light for 20 seconds. The resulting polymerized capsules were inspected under the bright field microscope and are shown in FIG. 15C and FIG. 15E.
The capsules in the second fraction were resuspended in an ice-cold 1×PBS buffer supplemented with 0.1% (w/v) Pluronic F-68 and 0.1% LAP, and transferred to room temperature (˜22° C.) for 15 min to induce the capsule swelling and expansion. Following the incubation at room temperature the capsules were photo-polymerized under 405 nm light for 20 seconds. The resulting capsules are shown in FIG. 15D and FIG. 15F.

In summary, the results presented in FIG. 15 prove that capsule size can be controlled by temperature, and more specifically that preincubation of capsules at temperature higher than 4° C., prior to the cross-linking step, leads to larger size capsules.

In addition to achieving the precise control over the capsule size, the shell thickness could be also tuned by adjusting the flow rates of the system or the concentration of the shell forming polymer. In one example, capsules generated using a microfluidics device 20 μm deep had a shell 2 μm thick (FIG. 14A). In another example, capsules generated using a microfluidics device 40 μm deep had a shell 3 μm thick (FIG. 14B). In yet another example, capsules generated using a microfluidics device 80 μm deep had a shell 5 μm thick (FIG. 14C).

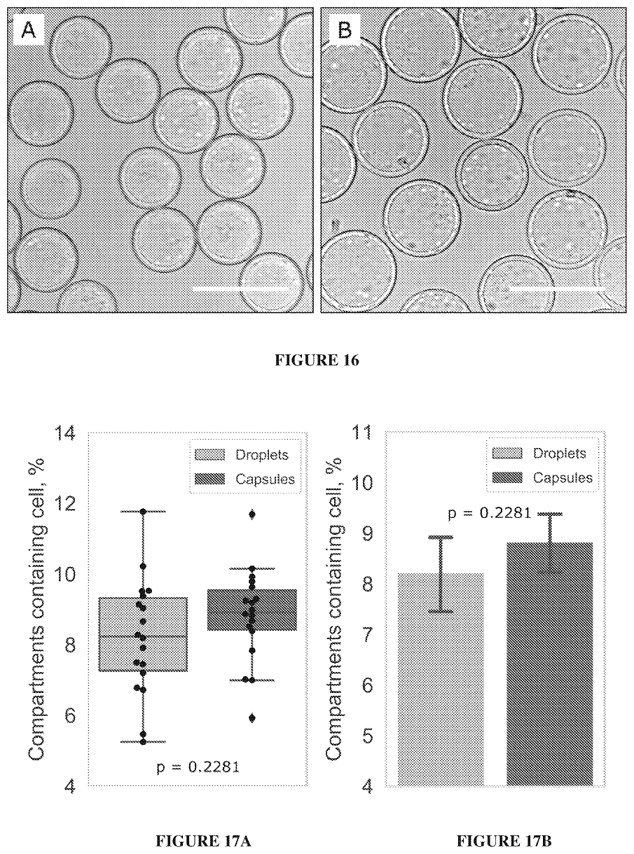

The shell thickness of the capsules could be also tuned by adjusting the concentration of the shell forming polymer. FIG. 16A shows 68 μm size capsules having 6.5 μm shell and 55 μm core. Such capsules can be generated by emulsifying 5% GMA with 15% dextran solutions followed by physical gelation and chemical cross-linking of the shell. As expected, pre-incubation at room temperature (˜22° C.) for 15 min before photo-polymerization increased the size of capsules to ˜82 μm diameter (70 μm core and 6 μm shell) as shown in FIG. 16B.

Altogether the examples presented above prove that capsule generation is highly flexible and is not restricted to one type of material. Capsule shell can be hardened using different agents such as temperature, light or chemicals. The capsule size and shell thickness can be tuned by changing the volumetric ratios of fluids during emulsification, the concentration of the ingredients in the liquid phases, the share force generated by carrier oil, or by changing the temperature at which capsules are generated/or processed. It should be understood that these examples are not limited.

To evaluate cell encapsulation and retention efficiency we used K-562 (ATCC, CCL-243) and HEK293 (ATCC, CRL-1573) cell lines. We quantified cell retention microscopically immediately after encapsulation, and then after capsule formation, and confirmed that the gel capsules efficiently retained encapsulated cells (FIG. 17). No significant difference was detected (two-tailored t-test (n=18) t=1.22, p=0.2281) confirming that compartmentalized cells were efficiently retained through all the steps of capsule generation. These results are in sharp contrast to PEGDA/Dextran composition, where majority of cells either moved to the shell phase, or to the shell/core interface (FIG. 3), or escaped compartmentalization (FIG. 4 and FIG. 5). As revealed in the examples below the gel capsules based on proteinaceous shell were compatible with multi-step biochemical and biological reactions; enabled passive exchange of low molecular weight compounds; retained large molecular weight compounds such as RNA or DNA inside; and the capsules were prone to user-controlled disintegration upon enzymatic treatment for releasing the internal capsule's content (e.g. encapsulated cell, PCR amplicon).

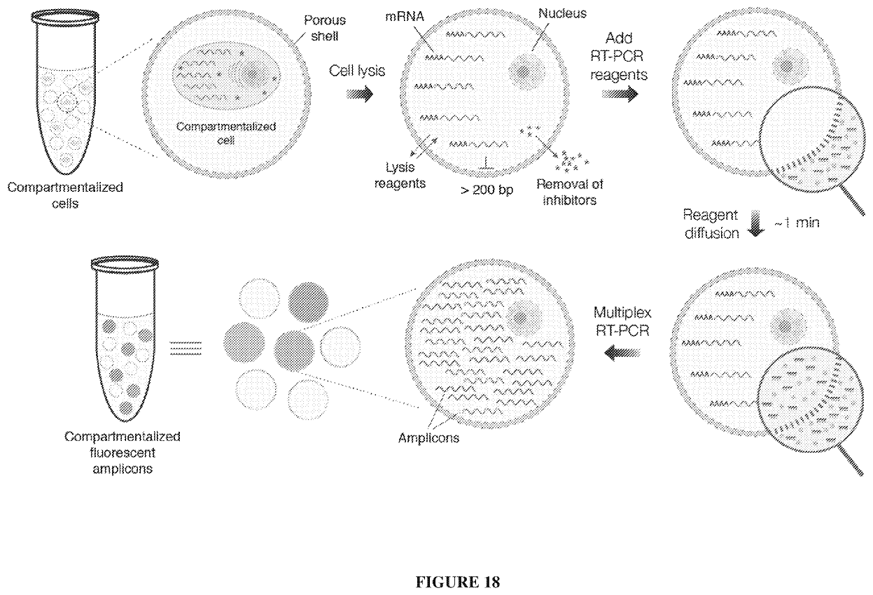

FIG. 18 shows a general concept of an invention for performing single-cell RT-PCR, but variations of this concept are discussed in the examples below and should be understood that extensions of this concept can be applied in single-cell genomics, single-cell transcriptomics, single-cell copy number variation, single-cell mutation, single-cell genomic aberration and cell-free nucleic acid analysis. In one of the examples, the individual cells are loaded in semi-permeable capsules and are lysed by dispersing capsules in a lysis buffer. Upon lysis the nucleic acid molecules longer than approximately 200 bp are preferably retained within the capsules, while smaller molecules (e.g. proteins, metabolites) are removed by washing capsules in washing buffer. Following lysis step, the capsules carrying single-cell lysate including nucleic acids are dispersed in assay mix containing fluorescently labelled oligonucleotides. The reagents quickly diffuse into the capsule enabling one-step and multi-step biochemical and enzymatic reactions to be conducted on encapsulated nucleic acid molecules. One particular example of such enzymatic reaction is RT-PCR assay. During the DNA amplification reaction, the fluorescently labelled oligonucleotides become incorporated into the PCR amplicons, turning an amplified DNA into a fluorescent product. Since oligonucleotides targeting different markers carries different fluorescent dyes emitting light at different wavelength it becomes possible to differentiate the gene expression based on the fluorescence signal. Therefore, using fluorescently labelled primers targeting different transcripts of interest the capsules acquire a fluorescence corresponding to the presence of the said transcript of interest, and thus enabling differentiation of encapsulated cells based on their transcriptional profile.

In the following, to showcase the use of capsules for single-cell RT-PCR and single-cell PCR assay we mainly used the capsules having diameter of approximately 75 μm with the shell being approximately 3 μm thick, however, for anyone experienced in the field it should be clear that other capsule sizes can be adapted for single-cell assays based on RT and PCR reactions.

### Example 2: Performing Single-Cell RT-PCR Using Semi-Permeable Capsules

To quantify the gene expression levels of individual cells it is appealing to consider the PCR assay with fluorescently labelled primers. Upon target amplification by PCR the fluorescent primers will be incorporated into amplicon making it highly fluorescent. Quantifying the fluorescent signal generated during the PCR would reflect the expression levels of a given transcript in a given cell. However, the use of fluorescently labelled primers is incompatible with qPCR assay and more generally with droplet microfluids assays since washing step is needed to remove the unincorporated DNA primers after post-PCR. To overcome this limitation others have applied hydrogel beads where one of the PCR primers grafted to the polymer mesh. During the PCR the amplicon remains covalently attached to the hydrogel mesh. For example, Kumaresan et al., showed emulsion PCR assay where nanoliter range droplets contain a bead covalently labelled with the reverse primer, fluorescent dye labelled forward primer, and a single target copy DNA. Following PCR, the fluorescent dye labelled forward primers are incorporated in double stranded product on the bead surface, the droplets are broken and the beads are analyzed by flow cytometry [36]. However, only 40% PCR efficiency was achieved for bead droplet PCR reducing the applicability of the method.

Herein, we reveal a novel method for individual cell profiling based on single-cell RT-PCR assay that uses fluorescent primers to target nucleic acid molecules of interest and does not rely on DNA primer incorporation into a hydrogel mesh. To identify the individual cells based on their gene expression profile, we performed a proof-of-concept study using a mixture of K562 and HEK293 cells. The cell lines were mixed at ratio 1:1 and encapsulated at a limiting dilution such that each capsule, on average, would contain no more than one cell. Cell encapsulation using droplet microfluidics is a well described process governed by Poisson statistics [37]. As a reference, we also separately encapsulated either K562 or HEK293 cells. After cell encapsulation, the water-in-oil droplets were converted to gel capsules using a two-step polymerization approach as detailed above. At first the emulsion droplets were incubated at 4° C. temperature to induce gelation of the shell, and then resulting capsules were dispersed in aqueous phase and cross-linked by photo-polymerization. Next, the capsules were suspended in GeneJET RNA Purification Kit Lysis Buffer (Thermo Scientific, K0732) containing 40 mM DTT and centrifuged immediately. The supernatant was aspirated and replaced with 1 mL of fresh lysis buffer followed by incubation at room temperature (21° C.) for 5 minutes. Following the incubation, the capsules were rinsed once in 1 mL lysis buffer and five-times in 1 mL washing buffer (10 mM Tris-HCl [pH 7.5] containing 0.1% (v/v) Triton X-100). During the washing steps the centrifugation was performed at 2000 g for 2 min.

Next, the genomic-DNA was depleted by adding 100 μl of close-packaged capsules to 200 μl reaction mix containing 0.05 U/μl DNAse I enzyme (Thermo Scientific, K2981) and 0.2 U/μl RNase Inhibitor (Thermo Scientific, EO0381) followed by incubation at 37° C. for 20 minutes. Next, 5 units of DNase I enzyme were added in a reaction mix and incubated at 37° C. for 10 minutes. After DNAse I treatment, the capsules were rinsed three-times in washing buffer (10 mM Tris-HCl [pH 7.5] containing 0.1% (v/v) Triton X-100) and subjected to a reverse transcription (RT) reaction.

The cDNA synthesis was performed in 200 μl reaction mix, containing 100 μl close-packaged capsules, 1×RT Buffer (Thermo Scientific, EP0751), 1× Oligo(dT)18 Primer (Thermo Scientific, SO131), 0.5 mM dNTP Mix (Thermo Scientific, R0192), 5 U/μl Maxima H Minus Reverse Transcriptase (Thermo Scientific, EP0751), 0.2 U/μl RiboLock RNase Inhibitor and incubated at 50° C. for 60 minutes. After cDNA synthesis, the capsules were rinsed three-times in washing buffer (10 mM Tris-HCl [pH 7.5] containing 0.1% (v/v) Triton X-100) and then subjected to polymerase chain reaction (PCR). The PCR was performed in 100 μl reaction volume by mixing 47 μl of closely-packed capsules with 53 μl of PCR reaction mix (Table 1).

TABLE 1
PCR mix composition

| | | |
|---|---|---|
| Volume | Material | Final |

| | | | |
|---|---|---|---|
| 50 | μl | 2x Phire Tissue Direct PCR Master | 1x |
| | | Mix | |
| 47 | μl | Close-packed capsules | — |
| 0.5 | μl | 100 μM 555-YAP-forward | 0.5 μM |
| 0.5 | μl | 100 μM YAP-reverse | 0.5 μM |
| 0.5 | μl | 100 μM 488- PTPRC -forward | 0.5 μM |
| 0.5 | μl | 100 μM PTPRC -reverse | 0.5 μM |
| 0.5 | μl | 100 μM 647-ACTB-forward | 0.5 μM |
| 0.5 | μl | 100 μM ACTB-reverse | 0.5 μM |
| 100 | μL | Final | — |

During the PCR, the specific markers preferentially expressed in HEK293, K562 or in both cell lines, were amplified. Specifically, the cDNA of YAP, PTPRC and ACTB markers, was amplified using marker specific primer set listed in Table 2. The primer set consisted of three primer pairs targeting the cDNA of YAP, PTPRC and ACT transcripts. Indeed, it should be understood that other markers can be targeted during the PCR step.

TABLE 2
The list of multiplex PCR primers used to amplify the markers of interest

| | | | Product |
|---|---|---|---|
| | Name | Sequence (5′-3′) | length |
| SEQ ID 1 | 555-YAP- | Alexa Fluor 555- | 584 bp |
| | forward | CCCTCGTTTTGCCATGAACC | |
| SEQ ID 2 | YAP-reverse | CCAGTGTTCCAAGGTCCACA | |
| SEQ ID 3 | 488-PTPRC- | Alexa Fluor 488- | 532 bp |
| | forward | ACATTGCTGCACAAGGTCCCAG | |
| SEQ ID 4 | PTPRC-reverse | AACCATCAGGCATCTCTGTCGC | |
| SEQ ID 5 | 647-ACTB- | Alexa Fluor 647- | 607 bp |
| | forward | ATTCCTATGTGGGCGACGA | |
| SEQ ID 6 | ACTB-reverse | AATGGTGATGACCTGGCCG | |

Each primer pair contained a sequence specific oligonucleotide, fluorescently labelled at 5′ end, that served as a forward primer. The reverse primer was not labelled with the fluorescent dye. Oligonucleotides targeting different markers carried different fluorescent dyes emitting light at different wavelength thus enabling differentiation of gene expression based on the fluorescence signal. During the PCR, the fluorescently labelled oligonucleotides were incorporated into the PCR amplicons, turning an amplified DNA into a fluorescent product. Although, as exemplified here, only forward primer contained a fluorescent dye, it should be understood to the expert in the field that it should be possible to use both reverse and forward primers labelled with a fluorescent dye and by doing so increase the fluorescence signal intensity of PCR amplicon. Target amplification was performed for 30 cycles with Phire Tissue Direct PCR Master Mix (Thermo Scientific, F170L) using thermocycling conditions provided in Table 3.

TABLE 3
The PCR thermocycling conditions

| Step | Temp | Time | | No Cycles |
|---|---|---|---|---|
| Initial | 98° C. | 5 | min | |
| denaturation | | | | |
| Amplification | 98° C. | 5 | s | 30 |
| | 64° C. | 5 | s | |
| | 72° C. | 20 | s | |
| Final extension | 72° C. | 1 | min | 1 |

After the PCR, 100 units of Exonuclease I (NEB, M0293L) enzyme was added directly to post-PCR mix, incubated at 37° C. for 15 minutes and rinsed three-times in washing buffer (10 mM Tris-HCl [pH 7.5] containing 0.1% (v/v) Triton X-100) to remove the excess of fluorescently labelled forward primers that have not been incorporated into PCR amplicons. The capsules then were analyzed using a fluorescence microscopy and flow cytometry.

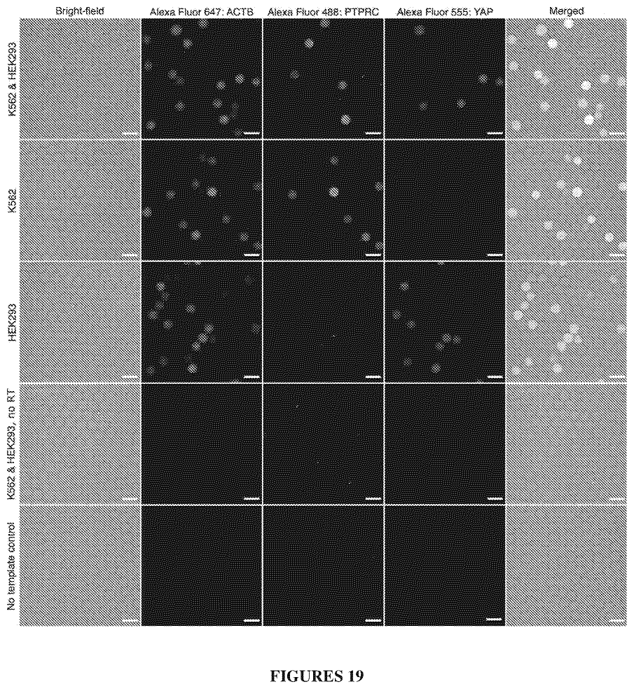

Given the differential expression of PTPCR and YAP markers in K562 and HEK293 cells, the capsules harbouring K562 cell should be positive in PTPCR marker, while capsules harbouring HEK293 cell should be YAP positive. In addition, both capsule types should be positive in ACTB marker since this gene is ubiquitously expressed in both cell types. Indeed, fluorescence microscopy analysis confirmed that capsules harbouring either K562 cells or HEK293 cells alone were distinguishable by expression of PTPRC or YAP gene marker, respectively (FIG. 19) and that both cells expressed ACTB. When capsules containing a mixture of cells were inspected under the fluorescence microscope, two distinct populations corresponding to K562 and HEK293 cells were detected based on either PTPRC-ACTB or YAP-ACTB positive counts (FIG. 19). Interestingly, a third population showing a fluorescent signal corresponding to ACTB target (FIG. 19) was also detected, indicating that capsule-based multiplex RT-PCR assay can detect the ambient RNA molecules originating from the lysed cells. Performing the same assay on empty capsules (no template control) as well as on capsules without reverse transcription enzyme but having K562 and HEK293 cells (no RT control), the ACTB positive capsules were not detected (FIG. 19).

### Example 3: Differentiating Live/Dead Cells and Performing Single-Cell RT-PCR Using Semi-Permeable Capsules

To differentiate whether the fluorescent RT-PCR signal originates from the ambient RNA molecules or from individual cells, we exemplify the use of fluorescent dye, DAPI (4′,6-diamidino-2-phenylindole), which binds to DNA and becomes fluorescent. Capsules that have a cell will contain DAPI-stained nuclei, while capsules that lack cells will also lack the DAPI-stained nuclei. Therefore, by recording the DAPI fluorescence it becomes possible to differentiate the source of RT-PCR signal: if the RT-PCR signal originates in the capsule that lacks DAPI-stained nucleus it implies that said capsule carries ambient nucleic acid(s), and if the RT-PCR signal originates in the capsule that contains DAPI-stained nucleus it implies that said capsule carries nucleic acid(s) from an encapsulated cell.

The cells were mixed at ratio 1:1 and encapsulated at a limiting dilution such that each capsule, on average, would contain no more than one cell. As a reference, K562 and HEK293 cells alone were encapsulated separately. After cell encapsulation, the capsules were washed in 1×PBS buffer containing 0.1% (w/v) Pluronic F-68. After washing the capsules were dispersed in 300 μL of PBS buffer, mixed with 700 μL of ice-cold 96% ethanol and transferred to −20° C. for at least 30-60 min incubation (although longer incubation times are also possible). Upon fixing the encapsulated cells with alcohol, the cell cytoplasm gets dehydrated and biomolecules such as DNA and RNA gets stabilized against the action by nucleases. Fixed cells can be stored at −20° C. for extended periods of time before proceeding to rehydration and RT-PCR assay.

After storage at −20° C., the tube with capsules was transferred on ice bucket and equilibrated for 5 minutes. Next, capsules were centrifuged at 2000 g for 2 minutes at 4° C. and washed once with 3×SSC buffer (Invitrogen, 15557044), supplemented with 0.04% BSA, 1 mM DTT and 0.2 U/μl RiboLock RNase Inhibitor (Thermo Scientific, EO0381). Next, cells were permeabilized by suspending the capsules in a buffer composed of 10 mM Tris-HCl [pH 7.5] supplemented with 0.3% (v/v) IGEPAL CA-630, 40 mM DTT and 10 mM EDTA, and incubated at room temperature for 15 minutes.

To stain cell nuclei, the capsules were immersed in 10 mM Tris-HCl [pH 7.5] buffer containing 0.1% (v/v) Triton X-100 and 5 μg/ml of DAPI (Invitrogen, D1306), and incubated in the dark, on ice for 15 minutes. Then capsules were rinsed five-times in 10 mM Tris-HCl [pH 7.5] buffer containing 0.1% (v/v) Triton X-100) and subjected to a reverse transcription (RT) reaction. The cDNA synthesis was performed in 200 μl reaction mix, containing 100 μl close-packed capsules resuspended in 1×RT Buffer (Thermo Scientific, EP0751), 1× Oligo(dT)18 Primer (Thermo Scientific, SO131), 0.5 mM dNTP Mix (Thermo Scientific, R0192), 5 U/μl Maxima H Minus Reverse Transcriptase (Thermo Scientific, EP0751), 0.2 U/μl RiboLock RNase Inhibitor and incubated at 50° C. for 60 minutes. After cDNA synthesis, the capsules were rinsed three-times in washing buffer (10 mM Tris-HCl [pH 7.5] containing 0.1% (v/v) Triton X-100) and then subjected to polymerase chain reaction (PCR).

The PCR was performed in 100 μl reaction volume by mixing 47 μl of closely-packed capsules with 53 μl of PCR reaction mix (Table 1). During the PCR, the specific markers preferentially expressed in HEK293 cells (YAP marker), K562 (PTPRC marker) or in both cell lines (ACTB marker), were amplified using marker specific primer set targeting the cDNA of YAP, PTPRC and ACTB transcripts (Table 2). Indeed, it should be understood that other markers can be targeted during the PCR step or during RT step. Each primer pair contained a sequence specific oligonucleotide, fluorescently labelled at 5′ end, that served as a forward primer (Table 2). The reverse primer was not labelled with the fluorescent dye. The oligonucleotides targeting different markers carried different fluorescent dyes emitting light at different wavelength, therefore, enabling differentiation of gene expression based on the fluorescence signal. During the PCR, the fluorescently labelled oligonucleotides were incorporated into the PCR amplicons, turning an amplified DNA into a fluorescent product. Although, as exemplified here, only forward primer contained a fluorescent dye, it should be understood to the expert in the field that it should be possible to use both reverse and forward primers labelled with a fluorescent dye and by doing so increase the fluorescence signal intensity of PCR amplicon. The PCR was performed for 40 cycles with Phire Tissue Direct PCR Master Mix (Thermo Scientific, F170L) using thermocycling conditions provided in Table 4.

TABLE 4
The PCR thermocycling conditions

| Step | Temp | Time | | No Cycles |
|---|---|---|---|---|
| Initial | 98° C. | 5 | min | |
| denaturation | | | | |
| Amplification | 98° C. | 5 | s | 40 |
| | 64° C. | 5 | s | |
| | 72° C. | 20 | s | |
| Final extension | 72° C. | 1 | min | 1 |

After the PCR, 100 units of Exonuclease I (NEB, M0293L) enzyme was added directly to post-PCR mix, incubated at 37° C. for 15 minutes and rinsed three-times in washing buffer (10 mM Tris-HCl [pH 7.5], 0.1% (v/v) Triton X-100) to remove the excess of fluorescently labelled forward primers that have not been incorporated into PCR amplicons. The capsules then were analyzed using a fluorescence microscopy.

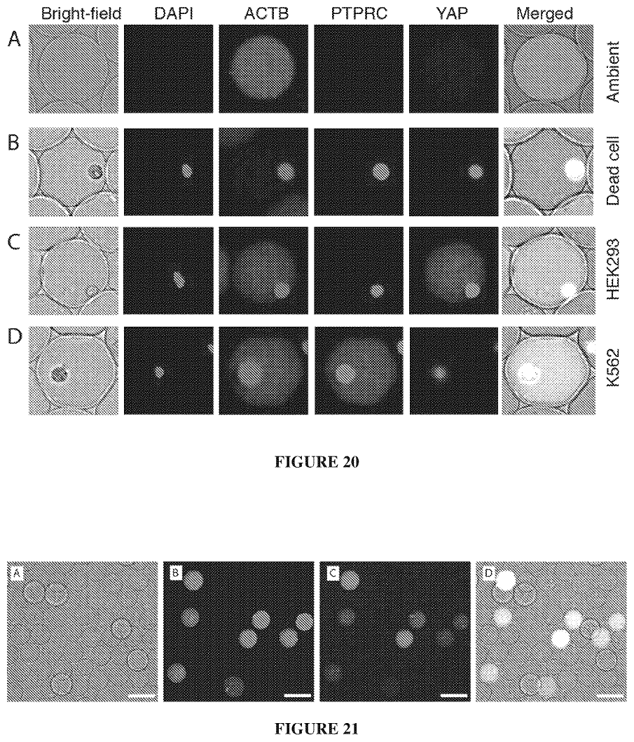

The capsules treated with a DNA fluorescent dye (DAPI) having emission wavelength different from those of PCR probes provides a simple approach to differentiate the capsules that contain fixed-cells (DAPI-positive) from those that do not (DAPI-negative). DAPI-stained cell nuclei would appear as highly fluorescent dots with emission maximum at the 461 nm (blue), whereas the fluorescent signal of RT-PCR product would be distributed within the capsule core (FIG. 20). Accordingly, the RT-PCR signal detected in DAPI-positive capsules would indicate the RNA molecules originating from the encapsulated cells; and the RT-PCR signal detected in DAPI-negative capsules would suggest the presence of ambient RNA molecules (e.g. found in the sample and/or reaction mix).

In addition, recording the fluorescence intensity and/or profile of RT-PCR product of ubiquitously expressed gene in DAPI-positive capsules could also serve as a useful readout for differentiating live and dead cells captured during the encapsulation step. Since dead cells contain compromised membranes and typically lack the cytoplasm RNA, therefore, in the event of capturing a dead cell, the capsule will show no characteristic RT-PCR signal (or the fluorescence signal will be weak) as compared to the event of capturing a live cell (FIG. 20).

The results shown in FIG. 20 indicate that staining ethanol fixed-cells with DNA intercalating dye such as DAPI prior conducting RT-PCR assay, enables identification of the capsules that during sample preparation and cell encapsulation step captured i) live-cells, ii) dead-cells or iii) cell-free (ambient) nucleic acid molecules.

### Example 4: Single-Cell PCR Using Semi-Permeable Capsules

To identify the individual cells based on genomic profile, we performed a proof-of-concept study using K562 cells. Cells were encapsulated at a limiting dilution such that each capsule, on average, would contain no more than one cell. The encapsulated cells were lysed by suspending capsules in lysis buffer (GeneJET RNA Purification Kit Lysis Buffer, Thermo Scientific, K0732) containing 40 mM DTT and centrifuged immediately. 900-1000 μL of supernatant was aspirated and replaced with 1000 μL of fresh lysis buffer, and incubated at the room temperature (˜21° C.) for 5 min. After incubation, the capsules were centrifuged and re-suspended again in the lysis buffer following centrifugation. Then, capsules were rinsed five-times in a washing buffer (10 mM Tris-HCl [pH 7.5] containing 0.1% (v/v) Triton X-100) and subjected to polymerase chain reaction (PCR). During the washing steps the centrifugation was performed at 2000 g for 2 min. The PCR was performed in 100 μl reaction volume by mixing 45 μl of closely-packed capsules with 55 μl of PCR reaction mix (Table 5).

TABLE 5
PCR mix composition

| | | |
|---|---|---|
| Volume | Material | Final |

| | | | |
|---|---|---|---|
| 50 | μl | 2x Phire Tissue Direct PCR Master Mix | 1x |
| 45 | μl | Close-packed capsules | — |
| 5 | μl | 10 μM 647-ACTB-forward/ACTB-reverse | 0.5 μM |
| | | primer mix | |
| 100 | μL | Final | — |

During the PCR, the ACTB gene was targeted and amplified using primers listed in Table 6, however, it should be understood that other DNA regions of interest, single nucleotide polymorphisms, DNA mutations, chromosome aberrations, etc., can be assayed during the PCR step.

TABLE 6
PCR primers used to amplify the region of interest of ACTB gene

| | | | Product |
|---|---|---|---|
| | Name | Sequence (5′-3′) | length |
| SEQ ID 5 | 647-ACTB- | Alexa Fluor 647- | 1040 bp |
| | forward | ATTCCTATGTGGGCGACGA | |
| SEQ ID 6 | ACTB-reverse | AATGGTGATGACCTGGCCG | |

The primer pair contained a sequence specific oligonucleotide, fluorescently labelled at 5′ end, that served as a forward primer. The reverse primer was not labelled with the fluorescent dye. During the PCR, the fluorescently labelled oligonucleotides were incorporated into the PCR amplicons, turning an amplified DNA into a fluorescent product. Although, as exemplified here, only forward primer contained a fluorescent dye, it should be understood to the expert in the field that it should be possible to use both reverse and forward primers labelled with a fluorescent dye and by doing so increase the fluorescence signal intensity of PCR amplicon. Target amplification was performed for 30 cycles with Phire Tissue Direct PCR Master Mix (Thermo Scientific, F170L) using thermocycling conditions provided in Table 3. After the PCR, 100 units of Exonuclease I (NEB, M0293L) enzyme was added directly to post-PCR mix, incubated at 37° C. for 15 minutes and rinsed three-times in washing buffer (10 mM Tris-HCl [pH 7.5] containing 0.1% (v/v) Triton X-100) to remove the excess of fluorescently labelled forward primers that have not been incorporated into PCR amplicons. The capsules then were analyzed using a fluorescence microscopy and are shown in FIG. 21. Results reveal that single-cell PCR it is possible to identify the cells carrying genomic region of interest became fluorescent.

### Example 5: RNA Molecule Amplification by Targeted RT-PCR Using Capsules

To identify and count the individual RNA molecules in a sample, we performed a proof-of-concept study using a mixture of total RNA purified from both K562 and HEK293 cells. The total RNA was mixed at ratio 1:1 (250:250 ng) in 100 μl of 15% (w/v) dextran (Sigma-Aldrich, D5251) and encapsulated alongside with 3% (w/v) GMA at a dilution such that each capsule, on average, would contain no more than one target RNA molecule (whereas target RNA molecules were chosen transcripts encoding YAP and PTPRC proteins). After sample encapsulation, the capsules were suspended in a washing buffer (10 mM Tris-HCl [pH 7.5], 0.1% (v/v) Triton X-100), rinsed twice and then 46 μl of closely-packed capsules transferred to a 0.2-ml reaction tube containing 54 μl of RT-PCR reagents and target specific oligonucleotides (Table 7).

TABLE 7
The RT-PCR reaction composition

| | | |
|---|---|---|
| Volume | Material | Final |

| | | | |
|---|---|---|---|
| 50 | μl | 2X Platinum SuperFi RT-PCR Master Mix | 1x |
| 1 | μl | SuperScript IV RT Mix | — |
| 46 | μl | Closely-packed capsules | — |
| 0.5 | μl | 100 μM 555-YAP-forward (SEQ ID 1) | 0.5 μM |
| 0.5 | μl | 100 μM YAP-reverse (SEQ ID 2) | 0.5 μM |
| 0.5 | μl | 100 μM 488-PTPRC -forward (SEQ ID 3) | 0.5 μM |
| 0.5 | μl | 100 μM PTPRC -reverse (SEQ ID 4) | 0.5 μM |
| 0.5 | μl | 100 μM 647-ACTB-forward (SEQ ID 5) | 0.5 μM |
| 0.5 | μl | 100 μM ACTB-reverse (SEQ ID 6) | 0.5 μM |
| 100 | μL | Final | — |

During the RT-PCR, the target RNA molecules encoding YAP and PTPRC proteins, as well as reference ACTB transcripts were converted to cDNA using gene specific primers that in turn also served in PCR as reverse primers (Table 2). The reverse primers in the reaction mix served as both the reverse transcription primers targeting specific mRNAs, and as the reverse primers for amplification of cDNA molecules by PCR. The forward primers targeted cDNA and were fluorescently labelled at 5′ end (Table 2). In addition, the forward primers specific to different cDNA targets carried fluorescent dyes emitting light at different wavelength to enable differentiation of amplified nucleic acid molecules based on the fluorescence signal alone. During the PCR step, the fluorescently labelled oligonucleotides along with other assay reagents diffused from the bulk into the core of the capsules and upon binding to the target DNA molecule got incorporated into the PCR amplicon thereby turning an amplified DNA into a fluorescent product. Although, as exemplified here, only forward primer contained a fluorescent dye, it should be understood to the expert in the field that it should be possible to use both reverse and forward primers labelled with a fluorescent dye and by doing so increase the fluorescence signal intensity of PCR amplicon. Target amplification was performed for 40 cycles with SuperScript™ IV One-Step RT-PCR System (Invitrogen, 12594100) using thermocycling conditions provided in Table 4.

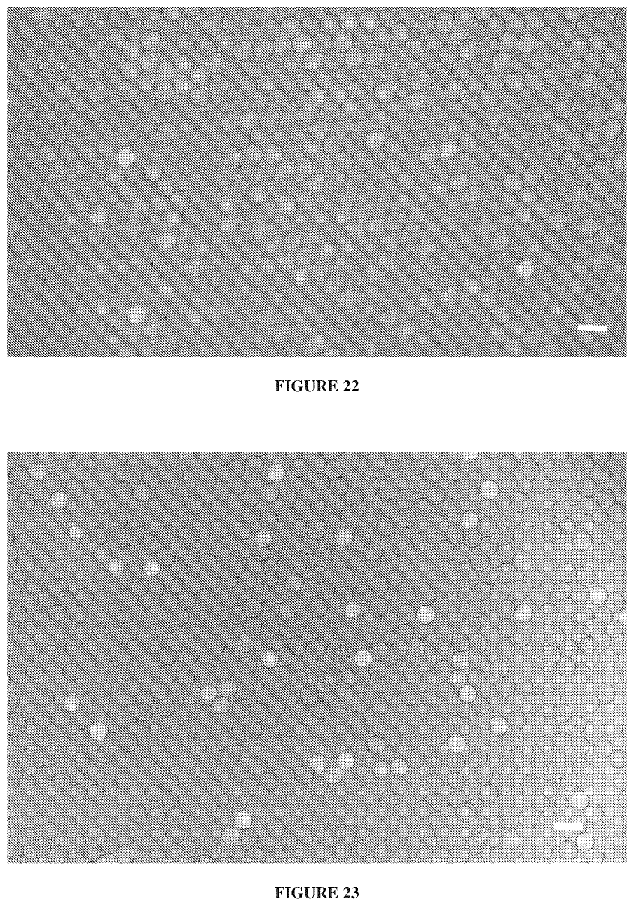

After the RT-PCR, 100 units of Exonuclease I (NEB, M0293L) enzyme was added directly to post-RT-PCR mix, incubated at 37° C. for 15 minutes and rinsed three-times in washing buffer (10 mM Tris-HCl [pH 7.5], 0.1% (v/v) Triton X-100) to remove the excess of fluorescently labelled forward primers that have not been incorporated into PCR amplicons. The capsules then were analyzed using a fluorescence microscopy and are shown in FIG. 22. It could be seen that individual capsules carrying ACTB, PTPRC or YAP transcripts became fluorescent in blue, green, red colors, respectively following RT-PCR assay, and therefore the RNA molecules present in biological sample could be identified and quantified.

### Example 6: DNA Molecule Amplification by PCR Using Capsules

To showcase individual nucleic acid molecule amplification by PCR and subsequent digital quantification, we performed a proof-of-concept study on DNA construct encoding green fluorescent protein (GFP). Specifically, ˜100 pg of pET29-GFP plasmid was added in 100 μl of 15% (w/v) dextran (Sigma-Aldrich, D5251) and encapsulated alongside with 3% (w/v) GMA at a dilution such that each capsule on average contains no more than one DNA molecule. After encapsulation and physical gelation steps, the capsules were suspended in a washing buffer (10 mM Tris-HCl [pH 7.5], 0.1% (v/v) Triton X-100), rinsed twice and then subjected to polymerase chain reaction (PCR). The PCR was performed in 100 μl reaction volume by mixing 45 μl of closely-packed capsules with 55 μl of PCR reaction mix (Table 8).

TABLE 8
The PCR reaction composition

| | | |
|---|---|---|
| Volume | Material | Final |

| | | | |
|---|---|---|---|
| 50 | μl | 2X Platinum SuperFi PCR Master Mix | 1x |
| 45 | μl | Close-packaged capsules | — |
| 5 | μl | 10 μM pET T7 forward/reverse primer | 0.5 μM |
| | | mix | |
| 100 | μL | Final | — |

During the PCR, the gene encoding green fluorescent protein of pET29-GFP plasmid was amplified using a specific set or oligonucleotides listed in Table 9. Indeed, it should be understood that other DNA templates or targets can be amplified during the PCR step.

TABLE 9
The sequence of PCR primers used to amplify the region of interest of
DNA construct

| | | | Product |
|---|---|---|---|
| | Name | Sequence (5′-3′) | length |
| SEQ ID 7 | pET T7 forward primer | GATCTTCCCCATCGGTGATGTCGGC | 1510 bp |
| SEQ ID 8 | pET T7 reverse primer | GCTGGCAAGTGTAGCGGTCACG | |

Target amplification was performed for 35 cycles with Platinum™ SuperFi™ PCR Master Mix (Invitrogen, 12358250) using thermocycling conditions provided in Table 10.

TABLE 10
The PCR thermocycling conditions

| Step | Temp | Time | | No Cycles |
|---|---|---|---|---|
| Initial | 98° C. | 30 | s | 1 |
| denaturation | | | | |
| Amplification | 98° C. | 5 | s | 35 |
| | 61° C. | 10 | s | |
| | 72° C. | 20 | s | |
| Final extension | 72° C. | 5 | min | 1 |

Next, the capsules were stained with 1×SYBR Green I (Invitrogen, S7563) at room temperature for 15 minutes, rinsed twice in washing buffer (10 mM Tris-HCl [pH 7.5], 0.1% (v/v) Triton X-100) and analyzed under fluorescence microscope.

The results presented in FIG. 23 show the capsules after PCR step. The capsules that carried individual DNA molecules and successfully amplified DNA during PCR became highly fluorescent upon staining with SYBR Green I dye. The capsules that lacked DNA or failed to amplify remained non-fluorescent.

### Example 7: Capsule Analysis with FACS Instrument

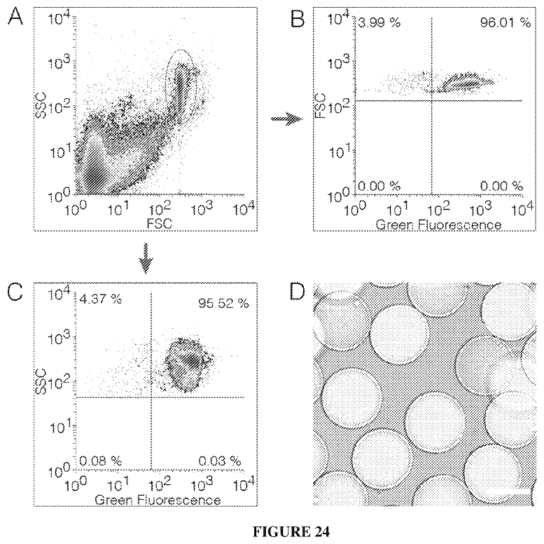

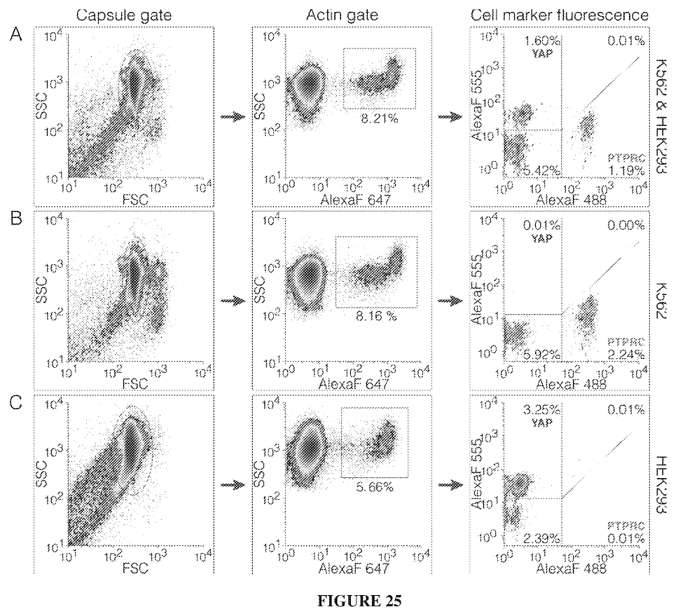

Using Partec CyFlow Space FACS instrument we first validated the capsule detection based on a signal derived from i) forward scatter, ii) side scatter and iii) fluorescence channels. As shown in FIG. 24 the capsules labelled with a fluorescent dye FITC-dextran 500K were successfully detected in all three channels. Next, we performed single-cell multiplex RT-PCR assay on capsules carrying K562 cells, HEK293 cells, or a mixture of both K562 and HEK293 cells following the experimental procedure described in Example 2, and subjected the capsules to flow cytometry. Up to 100,000 capsules were analyzed per experiment, although the total capsule count is not limited and could be easily scaled up. The distribution of flow cytometry events in the forward vs. side scatter plot (FIG. 25, Capsule gate), side scatter vs fluorescence of Alexa Fluor 647 plot (FIG. 25, ACTB gate) and Alexa Fluor 555 vs Alexa Fluor 488 intensity plot (FIG. 25, Cell marker gate) enabled precise quantification of cell type specific marker expression, identification of different cell types in a population, as well as ambient RNA and dead cells.

To perform a direct comparison between the flow cytometry and epifluorescence microscopy, we repeated the cell encapsulation and multiplex RT-PCR. Following the procedure described in Example 2 we prepared the capsules containing K562 alone, HEK293 alone, or a mixture of both K562 and HEK293 cells, and subjected them to multiplex RT-PCR. The capsules were washed to remove unincorporated DNA primers and divided into two fractions. One fraction was analyzed on FACS instrument and another fraction was analyzed under epifluorescence microscope. Results presented in Table 11 show that there is a very close agreement between microscopy and flow cytometry analysis.

TABLE 11
Comparison of the multiplex RT-PCR results using
flow cytometry and epifluorescence microscopy.

| | | | | | | | | |
|---|---|---|---|---|---|---|---|---|
| | | | | | | | | YAP & |
| | | | | YAP & | | PTPRC & | YAP & | PTPTC & |
| Sample | Total | ACTB | YAP | ACTB | PTPRC | ACTB | PTPTC | ACTB |
| ID | counts | positive | positive | positive | positive | positive | Positive | positive |

FACS analysis

| | | | | | | | | |
|---|---|---|---|---|---|---|---|---|
| K562 | 23619 | 2581 | 84 | 18 | 858 | 857 | 1 | 1 |
| | | (10.93%) | (0.36%) | (0.08%) | (3.63%) | (3.63%) | (<0.01%) | (<0.01%) |
| HEK293 | 19574 | 2828 | 1069 | 1012 | 1 | 1 | 6 | 6 |
| | | (14.45%) | (5.46%) | (5.17%) | (0.01%) | (0.01%) | (0.03%) | (0.03%) |
| K562 & | 31062 | 5776 | 940 | 862 | 524 | 516 | 6 | 5 |
| HEK293 | | (18.60%) | (3.03%) | (2.78%) | (1.69%) | (1.66%) | (0.02%) | (0.02%) |

Microscope analysis

| | | | | | | | | |
|---|---|---|---|---|---|---|---|---|
| K562 | 2822 | 285 | 3 | 3 | 90 | 90 | 2 | 2 |
| | | (10.1%) | (0.11%) | (0.11%) | (3.19%) | (3.19%) | (0.07%) | (0.07%) |
| HEK293 | 2128 | 272 | 103 | 102 | 0 | 0 | 0 | 0 |
| | | (12.78%) | (4.84%) | (4.79%) | (0.0%) | (0.0%) | (0.0%) | (0.0%) |
| K562 & | 2006 | 361 | 69 | 68 | 36 | 36 | 3 | 3 |
| HEK293 | | (18.0%) | (3.44%) | (3.39%) | (1.79%) | (1.79%) | (0.15%) | (0.15%) |

As expected the capsules carrying K562 cells were PTPRC positive (3.19-3.63% events), whereas the capsules carrying HEK293 cells were YAP positive (4.84-5.46% events). Capsules prepared with a mixture of HEK293 and K562 showed positive signal either in PTPRC or YAP channel, while K562 and HEK293 co-encapsulation events (2 cells of different type present in one capsule) were rare (˜0.02-0.15%).

The results presented in Table 11 also point that fraction of ACTB-positive events (3^{rd} column) was ˜3-fold higher than the number of capsules showing YAP (4^{th} column) or PTPRC (5^{th} column) signal. These results indicate that identification of encapsulated cells based on a highly expressed gene marker alone is prone to artifacts and thus should be avoided. This observation is also supported by previous reports where performing single-cell RT-PCR in water droplets led to a higher fraction of droplets being positive in an expression marker (e.g. CD45 marker) than the number of droplets carrying a cell [7, 9]. The release of mRNA during cell preparation and encapsulation process is likely source of these false-positive events. However, measuring fluorescence in two channels can facilitate identifying true positives events. For example, in the sample containing K562 cells 99.88% of PTPRC positive capsules were also positive in ACTB. In the sample containing HEK293 cells, 94.66% of YAP positive capsules were also positive in ACTB. (Table 11).

The flow cytometry results described here indicate that quantifying the absolute number of compartmentalized cells in a sample should not rely on a single expression marker alone due to false positive artifacts. Using two RNA markers (e.g. cell type specific gene marker and housekeeping gene marker) it is possible to precisely quantify the amount of specific cell types, yet such measurements are still limited as they do not reveal the absolute number of cells in a population (e.g. cell types expressing different set of markers), nor a proportion of a given cell type in the heterogeneous cell population. This analytical limitation could be particularly problematic for clinical sample analysis, which may contain dissociated cells with a compromised viability, or variable ratios of different cell types. We postulate that quantifying the absolute number of compartmentalized cells in a sample should rely on at least one marker that is not mRNA. We suggest that targeting biological molecules or structures of a different type (for example DNA, chromatin, lipids, etc.) should mitigate aforementioned artifacts and provide superior analytical resolution. One such appealing option would be to use a cell nucleus as a marker to identify functional micro-compartments (that is, capsules having a cell). We suggest that targeting two or more markers, such as (1) cell-type specific marker(s), and (2) mRNA independent marker(s) such as gDNA, nucleus, mitochondria, membrane or other cell feature common to all cells will enable absolute quantification of encapsulated cells in a population and identification of a particular cell type. Moreover, assay that would use three markers or more as a readout, for example, (1) one marker corresponding to ubiquitously expressed gene, (2) one or few markers corresponding to a cell type specific expression, and (3) mRNA independent marker such as gDNA, nucleus, mitochondria, membrane or other universal cell features, will enable absolute quantification of encapsulated cells in a population irrespectively of their type or physiological state, identification/quantification of viable and metabolically active cells, and quantification/identification of a particular cell type.

### Example 8: Long Term Storage of Encapsulated Cell and Nucleic Acids Preventing their Degradation

To evaluate whether or not, the capsules containing encapsulated species can be preserved for a long-term storage we tested capsule preservation in alcohol at −20° C. We performed a proof-of-concept study using a mixture of K562 and HEK293 cells. The cells were mixed at ratio 1:1 and encapsulated at a limiting dilution such that each capsule, on average, would contain no more than one cell. After cell encapsulation, the capsules were rinsed once in 1×PBS, containing 0.1% Pluronic F-68, and then once in 1×PBS. The capsules dispersed into three tubes:
1) Capsules in Tube 1 were preserved in alcohol as described below;
2) Capsules in Tube 2 were subjected to cell lysis, reverse transcription and only then preserved in alcohol as described below;
3) Capsules in Tube 3 were subjected to cell lysis, reverse transcription and PCR as described below (without preservation in alcohol).

Tube 1 (preservation of encapsulated cells): In this particular example, 300 μL of capsule suspension (100 μL of closely-packaged capsules and 200 μL of 1×PBS) were combined with 700 μL of ice-cold 96% ethanol and stored at −20° C. for at least 18 hours. Upon fixing the encapsulated cells within alcohol, the cell cytoplasm gets dehydrated and biomolecules such as DNA and RNA may be stabilized against the action by nucleases.

Following the incubation at −20° C., the tube with ethanol frozen cells in capsules was transferred on ice and equilibrated for 5 minutes. Next, capsules were centrifuged at 2000 g for 2 minutes at 4° C. and resuspended in 3×SSC buffer, supplemented with 0.04% BSA, 1 mM DTT and 0.2 U/μl RiboLock RNase Inhibitor (Thermo Scientific, EO0381). The capsules were briefly washed in Lysis Buffer (GeneJET RNA Purification Kit Lysis Buffer, Thermo Scientific, K0732), containing 40 mM DTT and resuspended in 1 mL of fresh Lysis Buffer followed by incubation at 21° C. (room temperature) for 5 min. Next, the capsules were centrifuged and re-suspended one more time in fresh Lysis Buffer. Finally, the capsules were rinsed five-times in a washing buffer (10 mM Tris-HCl [pH 7.5] containing 0.1% (v/v) Triton X-100 before proceeding to genomic DNA (gDNA) depletion reaction.

The gDNA was depleted by adding 100 μl of closely-packaged capsules to 200 μl reaction mix containing DNAse reaction mix (1× DNase I buffer, 0.05 U/l DNAse I enzyme (Thermo Scientific, K2981) and 0.2 U/μl RNase Inhibitor (Thermo Scientific, EO0381), followed by incubation at 37° C. for 20 minutes. Next, 5 units of DNase I enzyme were added in a reaction mix and capsules incubated at 37° C. for additional 10 minutes. After DNAse I treatment, the capsules were rinsed three-times in washing buffer (10 mM Tris-HCl [pH 7.5] containing 0.1% (v/v) Triton X-100) and subjected to a reverse transcription (RT) reaction.

The cDNA synthesis was performed in 200 μl RT reaction mix, containing 100 p close-packaged capsules, 1×RT Buffer, 1× Oligo(dT)18 Primers, 0.5 mM dNTP Mix, 5 U/μl Maxima H Minus Reverse Transcriptase, 0.2 U/μl RiboLock RNase Inhibitor. The reaction mix was incubated at 50° C. for 60 minutes. After cDNA synthesis, the capsules were rinsed three-times in washing buffer (10 mM Tris-HCl [pH 7.5], 0.1% (v/v) Triton X-100) and then subjected to polymerase chain reaction (PCR). See below.

Tube 2 (preservation of encapsulated nucleic acids). The capsules were briefly suspended washed in Lysis Buffer (GeneJET RNA Purification Kit Lysis Buffer, Thermo Scientific, K0732), containing 40 mM DTT, centrifuged at 2000 g for 2 min and resuspended in 1 mL of fresh Lysis Buffer followed by incubation at 21° C. (room temperature) for 5 min. Next, the capsules were centrifuged and re-suspended one more time in fresh Lysis Buffer. Finally, the capsules were rinsed five-times in a washing buffer (10 mM Tris-HCl [pH 7.5] containing 0.1% (v/v) Triton X-100 before proceeding to genomic DNA (gDNA) depletion.

The gDNA was depleted following the same procedure as used for Tube 1 described above. At first, 100 μl of closely-packaged capsules to 200 μl reaction mix containing DNAse reaction mix (1× DNase I buffer, 0.05 U/I DNAse I enzyme, 0.2 U/μl RNase Inhibitor), followed by incubation at 37° C. for 20 minutes. Next, 5 units of DNase I enzyme were added in a reaction mix incubated at 37° C. for additional 10 minutes. After DNAse I treatment, the capsules were rinsed three-times in washing buffer (10 mM Tris-HCl [pH 7.5] containing 0.1% (v/v) Triton X-100) and subjected to a RT reaction.

The cDNA synthesis was performed following the same procedure as used for Tube 1 described above. The 200 μl RT reaction mix, containing 100 μl close-packaged capsules, 1×RT Buffer, 1× Oligo(dT)18 Primers. 0.5 mM dNTP Mix, 5 U/μl Maxima H Minus Reverse Transcriptase, 0.2 U/μl RiboLock RNase Inhibitor. The reaction mix was incubated at 50° C. for 60 minutes. After cDNA synthesis, the capsules were rinsed three-times in washing buffer (10 mM Tris-HCl [pH 7.5], 0.1% (v/v) Triton X-100) and 300 μL of capsule suspension was mixed with 700 μL of ice-cold ethanol and stored at −20° C. for least 18 hours.

Following the incubation at −20° C. in alcohol, the capsules were equilibrated for 5 minutes on ice, centrifuged at 2000 g for 2 minutes at 4° C., washed three-times in washing buffer (10 mM Tris-HCl [pH 7.5], 0.1% (v/v) Triton X-100) and then subjected to polymerase chain reaction (PCR). See below.

Tube 3 (reference). The capsules were briefly suspended washed in Lysis Buffer (GeneJET RNA Purification Kit Lysis Buffer, Thermo Scientific, K0732), containing 40 mM DTT, centrifuged at 2000 g for 2 min and resuspended in 1 mL of fresh Lysis Buffer followed by incubation at 21° C. (room temperature) for 5 min. Next, the capsules were centrifuged and re-suspended one more time in fresh Lysis Buffer. Finally, the capsules were rinsed five-times in a washing buffer (10 mM Tris-HCl [pH 7.5] containing 0.1% (v/v) Triton X-100 before proceeding to genomic DNA (gDNA) depletion reaction.

The gDNA was depleted following the same procedure as used for Tube 1 and Tube 2 described above. At first, 100 μl of closely-packaged capsules to 200 p reaction mix containing DNAse reaction mix (1× DNase I buffer, 0.05 U/l DNAse I enzyme, 0.2 U/μl RNase Inhibitor), followed by incubation at 37° C. for 20 minutes. Next, 5 units of DNase I enzyme were added in a reaction mix incubated at 37° C. for additional 10 minutes. After DNAse I treatment, the capsules were rinsed three-times in washing buffer (10 mM Tris-HCl [pH 7.5] containing 0.1% (v/v) Triton X-100) and subjected to a RT reaction.

The cDNA synthesis was performed following the same procedure as used for Tube 1 and Tube 2 described above. The 200 μl RT reaction mix, containing 100 μl close-packaged capsules, 1×RT Buffer, 1× Oligo(dT)18 Primers. 0.5 mM dNTP Mix, 5 U/μl Maxima H Minus Reverse Transcriptase, 0.2 U/μl RiboLock RNase Inhibitor. The reaction mix was incubated at 50° C. for 60 minutes. After cDNA synthesis, the capsules were rinsed three-times in washing buffer (10 mM Tris-HCl [pH 7.5], 0.1% (v/v) Triton X-100) and then subjected to polymerase chain reaction (PCR). See below.

Performing PCR on capsules from Tube 1, Tube 2 and Tube 3.

The PCR was performed in 100 μl reaction volume by mixing 47 μl of closely-packed capsules with 53 μl of PCR reaction mix (Table 1). During the PCR, the specific markers preferentially expressed in HEK293, K562 or in both cell lines, were amplified. Specifically, the cDNA of YAP, PTPRC and ACTB markers, was amplified using marker specific primer set listed in Table 2. The primer set consisted of three primer pairs targeting the cDNA of YAP, PTPRC and ACTB transcripts. Indeed, it should be understood that other markers can be targeted during the PCR step. As described in above examples, each primer pair contained a sequence specific oligonucleotide, fluorescently labelled at 5′ end, that served as a forward primer. The reverse primer was not labelled with the fluorescent dye. During the PCR, the fluorescently labeled oligonucleotides were incorporated into the PCR amplicons, turning an amplified DNA into a fluorescent product. Target amplification was performed for 30 cycles with Phire Tissue Direct PCR Master Mix (Thermo Scientific, F170L) using thermocycling conditions provided in Table 3. After the PCR, 100 units of Exonuclease I (NEB, M0293L) enzyme was added directly to post-PCR mix, incubated at 37° C. for 15 minutes and rinsed three-times in washing buffer (10 mM Tris-HCl [pH 7.5] containing 0.1% (v/v) Triton X-100) to remove the excess of fluorescently labeled forward primers that have not been incorporated into PCR amplicons.

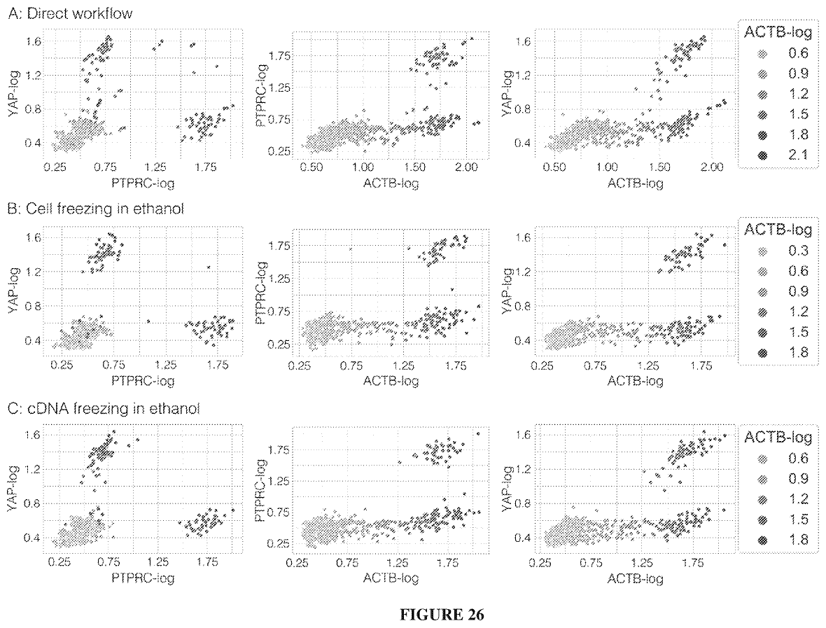

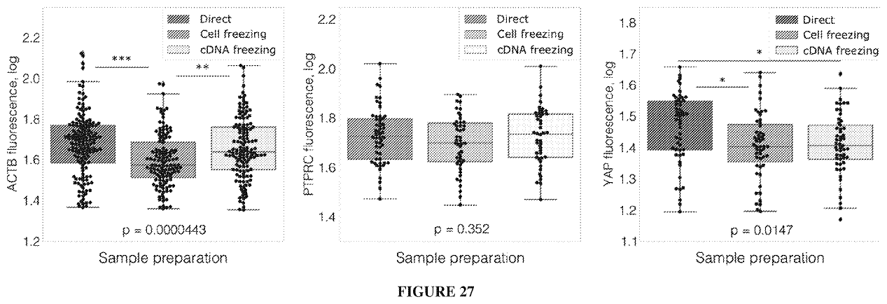

The post-PCR capsules images were recorded under the epifluorescence microscope and measured fluorescence intensities of ACTB, YAP and PTPRC amplicons were plotted for each condition as shown in FIG. 26. The nonparametric Kruskal-Wallis test was used to compare the intensity of positive capsules showing signal in ACTB, PTPRC, YAP and ACTB & PTPRC and ACTB & YAP and compared between preserved and non-preserved samples. The boxplots and calculated p values are shown in FIG. 27. All p values were higher than 0.05 indicating no statistically significant difference in sample distributions between processing techniques, irrespectively to analyzed cell marker, or their combination.

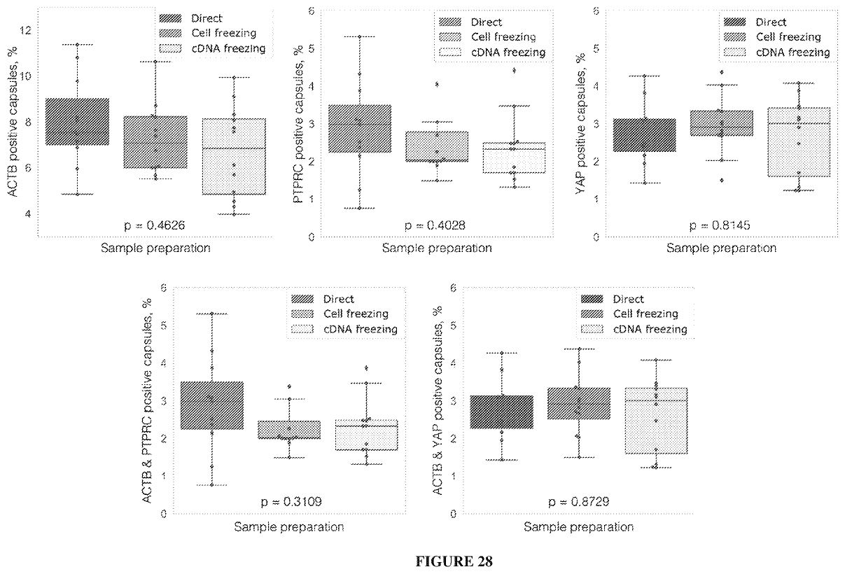

In addition, fluorescence intensity from ACTB, PTPRC and YAP-positive capsules was logarithmically transformed and then distribution normality was verified applying Lilliefors (Kolmogorov-Smirnov) normality test. Corresponding p values for ACTB, PTPRC and YAP fluorescence were 0.1118, 0.6766, 0.3761, which confirm a normal data distribution. The Levene's test for homogeneity of variance gave p values for ACTB, PTPRC and YAP fluorescence 0.2213, 0.9549 and 0.2439, respectively. Assuming equal variance between sample processing groups irrespectively to cell marker, one-way ANOVA and Tukey post hoc tests were applied (FIG. 28).

### Example 9: Nucleic Acid Retention in Capsules

To show nucleic acid molecule retention inside the microcapsules the following proof-of-concept experiment was conducted. The DNA fragment mix (GeneRuler 100 bp Plus DNA ladder (cat no. SM0321, ThermoFisherScientific) was encapsulated in water-in-oil droplets (˜70 μm size) as described above and processed as follow.

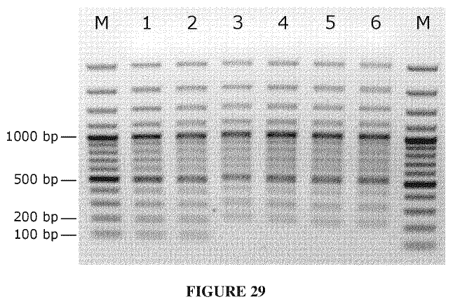

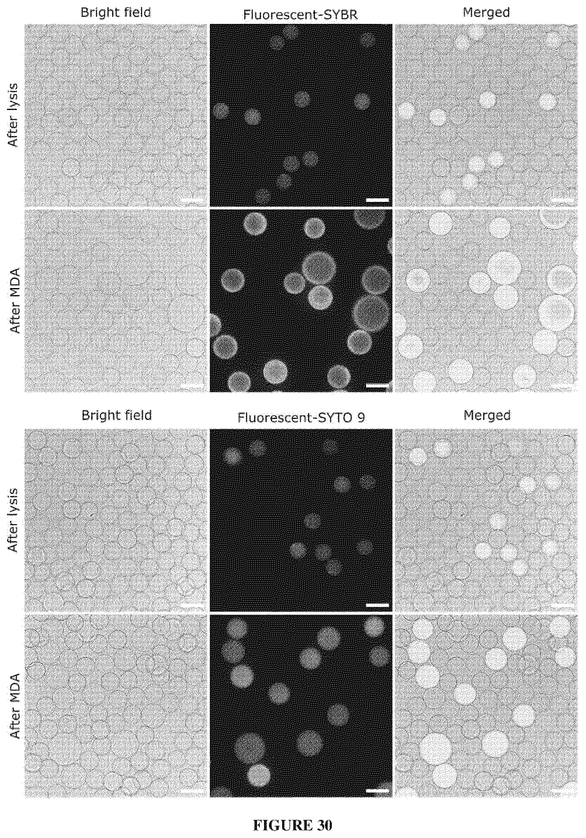

Condition 1: After encapsulation emulsion droplets were incubated at 4° C. for 30 min to induce solidification of liquid shell. Next, the microcapsules were released from emulsion and ˜20 μl of suspension comprising capsules was treated with 1 μl of Proteinase K (cat no. EO0491, ThermoFisherScientific) for 5 min at 50° C. Next, 20 μl of dissolved capsules were combined with 4 μl of 6×DNA Loading Dye (cat no. R0611, ThermoFisherScientific) and 20 μl sample volume was loaded on agarose well for DNA electrophoresis. The DNA electrophoresis results are shown in FIG. 29, well #1 (where wells #M corresponds to GeneRuler 100 bp Plus DNA ladder (cat no. SM0321, ThermoFisherScientific).

Condition 2: After encapsulation emulsion droplets were incubated at 4° C. for 30 min to induce solidification of liquid shell. Next, the microcapsules were released from emulsion into a washing buffer on ice, centrifuged at 2000 g at 4° C. and ˜20 μl of suspension comprising capsules was treated with 1 μl of Proteinase K, and loaded on agarose well for DNA electrophoresis as described above in “Condition 1”. The DNA electrophoresis results are shown in FIG. 29, well #2.

Condition 3: After encapsulation emulsion droplets were incubated at 4° C. for 30 min to induce solidification of liquid shell. Next, the microcapsules were released from emulsion into a washing buffer on ice supplemented with 0.1% LAP and immediately exposed to photopolymerization reaction under exposure to 405 nm LED device (Droplet Genomics, DG-BR-405) for 20 seconds. The capsules with cross-linked shell were incubated at room temperature (˜22° C.) for 30 min and then treated with Proteinase K, followed by DNA electrophoresis. The DNA electrophoresis results are shown in FIG. 29, well #3.

Condition 4: Microcapsules were prepared as described in “Condition 3”, however, the capsules with a cross-linked shell were incubated at 50° C. for 30 min and only then treated with Proteinase K, followed by DNA electrophoresis. The DNA electrophoresis results are shown in FIG. 29, well #4.

Condition 5: Microcapsules were prepared as described in “Condition 3”, however, the capsules with a cross-linked shell were first incubated at room temperature (˜22° C.) for 30 min (in the presence of dextranase enzyme, which hydrolyses the dextran constituting the core of the microparticles) and only then treated with Proteinase K, followed by DNA electrophoresis. The DNA electrophoresis results are shown in FIG. 29, well #5.

Condition 6: Microcapsules were prepared as described in “Condition 4”, however, the capsules with a cross-linked shell were first incubated at 50° C. for 30 min (in the p ene of dextranase enzyme, which hydrolyses the dextran constituting the core of the microparticles) and only then treated with Proteinase K, followed by DNA electrophoresis. The DNA electrophoresis results are shown in FIG. 29, well #6.

The results presented in FIG. 29 show that no detectable difference was observed in retention between samples with and without dextranase treatment and that capsules efficiently retain 200 bp and larger DNA fragments, while 100 bp DNA fragments can passively move from the capsules and thus are not retained (wells #3-6). There is no preferential DNA fragment loss during encapsulation process (well #1).

### Example 10: Whole-Genome Amplification

The K-562 cells were encapsulated at a limiting dilution such that each capsule, on average contained no more than one cell. Specifically, K-562 cells were suspended in 15% (w/v) dextran, MW 500 k at dilution ˜200 k cells/100 uL and co-encapsulated with 3% (w/v) GMA solution in 1×PBS using a microfluidics chip 40 μm height and having a nozzle 40 μm wide. Flow-rates used were: 3% GMA solution—250 μL/h, 15% dextran solution with cells—100 μL/h and the carrier oil—700 μL/h. Encapsulation was performed at room temperature (21-22° C.) for 20-25 minutes. Emulsions were collected in 1.5 mL tube, prefilled with 200 μL of light mineral oil. After encapsulation, emulsions were immediately transferred at 4° C. for 30 minutes to solidify the shell. Continuing procedures on ice, the intermediate-microcapsules were recovered from the emulsion using commercial emulsion breaker (Droplet Genomics, DG-EB-1) and resuspended 1×DPBS buffer supplemented with 0.1% Pluronic F-68. Microcapsule suspension was mixed with 0.1% (w/v) LAP and exposed to 405 nm photo-illumination using LED device (Droplet Genomics, DG-BR-405) for 20 seconds. After cross-linking step, the microcapsules were suspended in GeneJET RNA Purification Kit Lysis Buffer (Thermo Scientific, K0732), containing 40 mM DTT and centrifuged. 900-1000 μL of supernatant was aspirated and replaced with 1 mL of fresh lysis buffer followed by incubation at room temperature for 5 min. After the incubation microcapsules were centrifuged and re-suspended again in the lysis buffer followed by centrifugation. The capsules were rinsed five-times in a washing buffer (10 mM Tris-HCl [pH 7.5] (Invitrogen, 15567027) containing 0.1% (v/v) Triton X-100 (Thermo Scientific, 85111) and subjected to multiple displacement amplification (MDA) reaction. During the washing steps the centrifugation was performed at 1000-2000 g for 2 min.

The MDA was performed in 100 μL reaction volume by mixing 50 μL of closely-packed capsules with 50 μl of MDA reaction mix containing 1× Reaction Buffer (Thermo Scientific, EP0091), 1 mM dNTP Mix (Invitrogen, 18427013), 25 μM Exo-Resistant Random Primer (Thermo Scientific, SO181), 1 mM DTT (Thermo Scientific, R0861) and 0.5 U/μL phi29 DNA Polymerase (Thermo Scientific, EP0091).

Whole genome amplification (WGA) by MDA reaction was performed at 30° C. for 6 hours. After the WGA, the microcapsules were rinsed once in 1 mL of 10 mM Tris-HCl [pH 7.5] buffer containing 0.1% (v/v) Triton X-100 and 5 mM EDTA (Invitrogen, 15575020). Then, capsules were rinsed twice in 10 mM Tris-HCl [pH 7.5] buffer containing 0.1% (v/v) Triton X-100. Post-MDA capsules were stained with 1×SYBR Green I (Invitrogen, S7585) and 5 μM SYTO 9 (Invitrogen, S34854) for 30 minutes at room temperature, then rinsed twice in 10 mM Tris-HCl [pH 7.5] buffer containing 0.1% (v/v) Triton X-100 and analyzed using a fluorescence microscopy.

The results presented in FIG. 30 show that individual genomes were successfully amplified in microcapsules comprising cells. Interestingly, upon WGA some capsules swelled and expanded in size, presumably due to increased osmotic pressure exerted on the microcapsule shell.

All publications, patents, and patent applications mentioned herein are hereby incorporated by reference in their entirety. In cases where the present specification and a document incorporated by reference include conflicting and/or inconsistent disclosure, the present specification shall control.

### REFERENCES

1. Joensson, H. N. and H. Andersson Svahn, Droplet microfluidics—a tool for single-cell analysis. Angew Chem Int Ed Engl, 2012. 51(49): p. 12176-92.
2. Matula, K., F. Rivello, and W. T. S. Huck, Single-Cell Analysis Using Droplet Microfluidics. Advanced Biosystems, 2020. 4(1).
3. Chen, Q., et al., Controlled assembly of heterotypic cells in a core-shell scaffold: organ in a droplet. Lab Chip, 2016. 16(8): p. 1346-9.
4. Zhu, Z., et al., Highly sensitive and quantitative detection of rare pathogens through agarose droplet microfluidic emulsion PCR at the single-cell level. Lab on a Chip, 2012. 12(20): p. 3907-3913.
5. White, A. K., et al., High-throughput microfluidic single-cell RT-qPCR. Proc Natl Acad Sci USA, 2011. 108(34): p. 13999-4004.
6. Gong, Y. A., A. O. Ogunniyi, and J. C. Love, Massively parallel detection of gene expression in single cells using subnanolitre wells. Lab on a Chip, 2010. 10(18): p. 2334-2337.
7. Eastburn, D. J., A. Sciambi, and A. R. Abate, Ultrahigh-Throughput Mammalian Single-Cell Reverse-Transcriptase Polymerase Chain Reaction in Microfluidic Drops. Analytical Chemistry, 2013. 85(16): p. 8016-8021.
8. Pellegrino, M., et al., High-throughput single-cell DNA sequencing of acute myeloid leukemia tumors with droplet microfluidics. Genome Res, 2018. 28(9): p. 1345-1352.
9. Kim, S. C., et al., Single-Cell RT-PCR in Microfluidic Droplets with Integrated Chemical Lysis. Analytical Chemistry, 2018. 90(2): p. 1273-1279.
10. Zhang, H. F., et al., Massively Parallel Single-Molecule and Single-Cell Emulsion Reverse Transcription Polymerase Chain Reaction Using Agarose Droplet Microfluidics. Analytical Chemistry, 2012. 84(8): p. 3599-3606.
11. Rakszewska, A., et al., Quantitative Single-Cell mRNA Analysis in Hydrogel Beads. Angew Chem Int Ed Engl, 2016. 55(23): p. 6698-701.
12. Lan, F., et al., Single-cell genome sequencing at ultra-high-throughput with microfluidic droplet barcoding. Nat Biotechnol, 2017. 35(7): p. 640-646.
13. Spencer, S. J., et al., Massively parallel sequencing of single cells by epic PCR links functional genes with phylogenetic markers. ISME J, 2016. 10(2): p. 427-36.
14. Leonaviciene, G., et al., Multi-step processing of single cells using semi-permeable capsules. Lab Chip, 2020. 20(21): p. 4052-4062.
15. Pregibon, D. C. and P. S. Doyle, Optimization of encoded hydrogel particles for nucleic acid quantification. Anal Chem, 2009. 81(12): p. 4873-81.
16. Lewis, C. L., et al., Fabrication of uniform DNA-conjugated hydrogel microparticles via replica molding for facile nucleic acid hybridization assays. Anal Chem, 2010. 82(13): p. 5851-8.
17. Mitra, R. D. and G. M. Church, In situ localized amplification and contact replication of many individual DNA molecules. Nucleic Acids Res, 1999. 27(24): p. e34.
18. Rieger, C., et al., Polony analysis of gene expression in ES cells and blastocysts. Nucleic Acids Res, 2007. 35(22): p. e151.
19. Walser, M., et al., Novel method for high-throughput colony PCR screening in nanoliter-reactors. Nucleic Acids Res, 2009. 37(8): p. e57.
20. Bigdeli, S., et al., A simple method for encapsulating single cells in alginate microspheres allows for direct PCR and whole genome amplification. PLoS One, 2015. 10(2): p. e0117738.
21. Brangwynne, C. P., P. Tompa, and R. V. Pappu, Polymer physics of intracellular phase transitions. Nature Physics, 2015. 11(11): p. 899-904.
22. Thorsen, T., et al., Dynamic pattern formation in a vesicle-generating microfluidic device. Phys Rev Lett, 2001. 86(18): p. 4163-6.
23. Garstecki, P., et al., Formation of monodisperse bubbles in a microfluidic flow-focusing device. Applied Physics Letters, 2004. 85(13): p. 2649-2651.
24. Sugiura, S., M. Nakajima, and M. Seki, Prediction of Droplet Diameter for Microchannel Emulsification. Langmuir, 2002. 18(10): p. 3854-3859.
25. Ward, T., et al., Microfluidic flow focusing: Drop size and scaling in pressure versus flow-rate-driven pumping. Electrophoresis, 2005. 26(19): p. 3716-3724.
26. Anna, S. L., N. Bontoux, and H. A. Stone, Formation of dispersions using “flow focusing” in microchannels. Applied Physics Letters, 2003. 82(3): p. 364-366.
27. Nie, Z., et al., Emulsification in a microfluidic flow-focusing device: effect of the viscosities of the liquids. Microfluidics and Nanofluidics, 2008. 5(5): p. 585-594.
28. Zheng, B., J. D. Tice, and R. F. Ismagilov, Formation of Droplets of Alternating Composition in Microfluidic Channels and Applications to Indexing of Concentrations in Droplet-Based Assays. Analytical Chemistry, 2004. 76(17): p. 4977-4982.
29. Teh, S.-Y., et al., Droplet microfluidics. Lab on a Chip, 2008. 8(2).
30. Jo, Y. K. and D. Lee, Biopolymer Microparticles Prepared by Microfluidics for Biomedical Applications. Small, 2020. 16(9).
31. Holtze, C., et al., Biocompatible surfactants for water-in-fluorocarbon emulsions. Lab on a Chip, 2008. 8(10).
32. Vijayakumar, K., et al., Rapid cell extraction in aqueous two-phase microdroplet systems. Chemical Science, 2010. 1(4): p. 447-452.
33. Van Den Bulcke, A. I., et al., Structural and rheological properties of methacrylamide modified gelatin hydrogels. Biomacromolecules, 2000. 1(1): p. 31-8.
34. Gasperini, L., J. F. Mano, and R. L. Reis, Natural polymers for the microencapsulation of cells. J R Soc Interface, 2014. 11(100): p. 20140817.
35. Link, D. R., et al., Geometrically mediated breakup of drops in microfluidic devices. Phys Rev Lett, 2004. 92(5): p. 054503.
36. Kumaresan, P., et al., High-throughput single copy DNA amplification and cell analysis in engineered nanoliter droplets. Analytical Chemistry, 2008. 80(10): p. 3522-3529.
37. Mazutis, L., et al., Single-cell analysis and sorting using droplet-based microfluidics. Nat Protoc, 2013. 8(5): p. 870-91.

1.-55. (canceled)

56. A method of producing a microcapsule encapsulating a biological entity, wherein the resulting microcapsule comprises a semi-permeable shell surrounding a core, wherein the biological entity is in the core, the method comprising:
(a) forming a water-in-oil droplet comprising a first solute, a second solute and the biological entity, wherein the first solute is a polyampholyte with or without an antichaotropic agent, and the second solute is a polyhydroxy compound and/or antichaotropic agent, wherein the polyampholyte comprises one or more covalently cross-linkable groups and wherein aqueous phase separation occurs inside the water-in-oil droplet into a shell phase enriched in the first solute and a core phase enriched in the second solute;
(b) induces gelation and/or precipitation of the shell phase to form an intermediate product with a solidified shell; and
(c) forming intermolecular covalent cross-links with the one or more covalently cross-linkable groups to form the microcapsule comprising a semi-permeable shell of covalently cross-linked polyampholyte and a core, wherein the biological entity is in the core.

57. The method according to claim 56, wherein water-in-oil droplets are generated in a microfluidic device or other device, assembly or instrument capable of forming a water-in-oil droplet, such as a glass capillary device.

58. The method according to claim 56, wherein (a) is performed by mixing a first solution comprising the first solute with a second solution comprising the second solute, and simultaneously or separately combining the mixture of the first and second solutions with the carrier oil having a surfactant, wherein the biological entity is dispersed in one of the solutions, preferably in the second solution, and/or wherein the biological entity dispersed in a third solution which is mixed with the first solution and the second solution to form the mixture.

59. The method according to claim 58, wherein the first solution comprises 0.1 to 20% (w/v) of the polyampholyte, preferably below 20% and more preferably in the range of 1 to 15% (w/v).

60. The method according to claim 56, wherein the second solution comprises 0.1 to 40% (w/v) of the polyhydroxy compound, and preferably in the range of 3 to 30% (w/v).

61. The method according to claim 56, wherein the first and/or second solution comprises an antichaotropic agent at a concentration of 0.01 to 2 M, preferably 0.5 to 1.2 M.

62. The method according to claim 56, wherein the polyampholyte is modified with one or more chemically cross-linkable groups for the covalent cross-linking in (c), wherein the polyampholyte has the average number of substitutions ranging from 10 to 90%, preferably 40 to 90%, more preferably 60 to 80%.

63. The method according to claim 56, wherein the first solute comprising polyampholyte and the second solute comprising polyhydroxy compound, phase separates in response to salts, temperature change, pH change or ionic change of a solvent.

64. The method according to claim 59, wherein the polyampholyte:
(a) is a biopolymer, a modified biopolymer or a synthetic polymer, wherein the modified biopolymer is the polyampholyte modified with one or more chemically cross-linkable groups for the covalent cross-linking in (c), wherein the polyampholyte has a degree of substitution of 10 to 90%, preferably 40 to 90%, more preferably 60 to 80%;
(b) comprises peptide bonds; and/or
(c) is a peptide, a polypeptide, an oligopeptide or a protein.

65. The method according to claim 59, wherein the polyampholyte is a peptide, a polypeptide, an oligopeptide or a protein, and wherein at least 10% of the amino acids in the polyampholyte are disorder-promoting amino acids, preferably wherein at least 30% of the amino acids in the polyampholyte are disorder-promoting amino acids, where the disorder promoting amino acids are selected from proline, glycine, glutamic acid/glutamate, serine, lysine, alanine, arginine, and glutamine.

66. The method according to claim 59, wherein the polyampholyte belongs to extracellular matrix proteins, proteoglycans, glycosaminoglycans, or a hydrolyzed form of any of the foregoing, and preferably wherein the extracellular matrix protein is collagen, or a hydrolyzed form thereof, such as gelatin.

67. The method according to claim 60, wherein the polyhydroxy compound is selected from a polyelectrolyte, polysaccharide, a carbohydrate, an oligosaccharide or a sugar, which can be natural or synthetic.

68. The method according to claim 67, wherein the polyhydroxy compound is one or more of glucan, dextran, dextrin, natural gum, alginate, cellulose, hemicellulose, starch selected from amylose or amylopectin, agarose, agar-agar, chitin, hyaluronic acid, heparin, pectin, chitosan, curdian, pullulan, inulin, graminan, levan, polyglycerol.

69. The method according to claim 67, wherein the polyhydroxy compound is a synthetic polymer, and optionally wherein the synthetic polymer is a branched polysaccharide.

70. The method according to claim 60, wherein the polyhydroxy compound has a molecular weight between 300 Da to 5000 kDa, preferably greater than 10 kDa, and even more preferably greater than 100 kDa.

71. The method according to claim 56, wherein the produced microcapsule is between 1 μm and 1000 μm in diameter, preferably 10 μm to 500 μm in diameter, and more preferably 50 μm to 200 μm.

72. The method according to claim 56, wherein the semi-permeable shell is permeable to compounds having molecular weight of 120,000±80,000 Da or less through the shell and is mostly impermeable to compounds having molecular weight of 300,000 Da±100,000 Da and above.

73. The method according to claim 56, wherein the water-in-oil droplets are formed in a fluorinated, perfluorinated, hydrocarbon or synthetic continuous oil phase.

74. The method according to claim 56, wherein the polyampholyte is a thermo-responsive polymer, and wherein changing the temperature of the water-in-oil droplet induces physical gelation of the thermo-responsive polymer to achieve solidification in the shell phase to form the intermediate microcapsule, wherein the solidified gel is a thermo-reversible gel.

75. The method according to claim 56, wherein the temperature is changed to a temperature from below 40° C. to at or above 0° C., and preferably below 25° C., and more preferably at 4° C.

76. The method according to claim 56, wherein after incubating and prior to (c) the carrier oil is replaced by an aqueous solution.

77. The method according to claim 76, wherein the replacement occurs by demulsification using an emulsion destabilizing agent.

78. The method according to claim 56, wherein (c) comprises exposing the intermediate microcapsule to a chemical agent, irradiation, or heat, or any combination thereof, to covalently cross-link the polyampholyte.

79. The method according to claim 56, wherein (c) comprises activating the chemically cross-linkable groups by exposing the intermediate microcapsule to an initiator, such as chemical-initiator, such as tetramethylethylenediamine, ammonium persulfate, photo-initiator, such as lithium phenyl-2,4,6-trimethylbenzoylphosphinate, thermal initiator, such as heat, radiative-initiator, such as visible or UV light, or any combination thereof.

80. The method according to claim 56, wherein (c) comprises covalently cross-linking by photo-polymerisation.

81. The method according to claim 56, comprising encapsulating a plurality of biological entities in a plurality of microcapsules, wherein in (a) at least 1% of the water-in-oil droplets comprise a single biological entity and more preferably where at least 10% of the water-in-oil droplets each comprise a single biological entity.

82. The method of claim 81, wherein the single biological entity is a single cell.

83. A method of performing one or more reactions on a biological entity, the method comprising:
(i) isolating the biological entity in a microcapsule using the method of claim 56;
(ii) performing the one or more reactions on the biological entity in the microcapsule;
(iii) optionally: breaking the semi-permeable shell of the microcapsule by cleaving peptide bonds using one or more proteases.

84. The method of claim 83, wherein (ii) comprises suspending the microcapsules in an aqueous reaction mix comprising one or more components for performing the reaction, and allowing the one or more components to come into contact with the biological entity by diffusion from the aqueous reaction mix into the microcapsule, wherein the one or more components are one or more reagents, one or more proteins, one or more enzymes, and/or one or more substrates.

85. The method of claim 84, wherein the biological entity is a cell and the one or more reactions comprises cell lysis to release the desired component from the cell, optionally wherein the desired component is nucleic acid.

86. The method of claim 84, wherein the one or more reactions comprises nucleic acid analysis, wherein the nucleic acid analysis comprises one or more of reverse transcription (RT), transcription, and nucleic acid amplification.

87. The method of claim 86, wherein the biological entity is a cell and (ii) comprises lysing the cell and performing nucleic acid amplification on one or more nucleic acids released from the cell by RT-PCR to generate fluorescently labelled DNA by incorporating fluorescently labelled DNA oligonucleotides into newly synthesized DNA, or staining amplified DNA with a fluorescent probe or dye.

88. The method of claim 86, wherein the biological entity is a cell and (ii) comprises lysing the cell and performing nucleic acid amplification on one or more nucleic acids released from the cell by RT-PCR to generate fluorescently labelled DNA by hybridizing the newly synthesized DNA with fluorescently labelled DNA oligonucleotides directly or indirectly via an additional DNA oligonucleotide.

89. The method of claim 87, comprising digitally recording the fluorescence and/or sorting the microcapsules using a Fluorescence Activated Cell Sorting (FACS) instrument.

90. A kit for making the microcapsule according to the method of claim 56, the kit comprising:
(a) a polyhydroxy compound;
(b) optionally: an antichaotropic agent;
(c) a polyampholyte comprising one or more covalently cross-linkable groups;
(d) a microfluidic chip; and
(e) a carrier oil with or without a surfactant.

91. The kit according to claim 90, wherein:
(i) the antichaotropic agent is a kosmotropic salt such as a sulphate, a phosphate or a citrate, and/or the polyhydroxy compound is selected from a polyelectrolyte, polysaccharide, a carbohydrate, an oligosaccharide or a sugar, which can be natural or synthetic; and/or
(ii) the polyampholyte is a biopolymer, a chemically modified biopolymer or a synthetic polymer that comprises peptide bonds that can be covalently cross-linked upon a reaction with chemical agents, free radicals, photo-initiator, or else polymerize under irradiation and/or heat;
(iii) the microfluidics chip is configured to produce water-in-oil droplets;
(iv) the carrier oil comprises one or more stabilizing surfactants wherein the polyampholyte described in (ii) wets the interface of the water-in-oil droplets; and
(v) the antichaotropic agent and/or polyhydroxy compound and polyampholyte are chosen such that when combined, the antichaotropic agent and/or polyhydroxy compound phase separate from the polyampholyte.

92. The kit according to claim 90, wherein the polyampholyte is provided in a first solution and/or wherein the polyhydroxy compound and/or the antichaotropic agent is provided in a second solution.

93. The kit according to claim 90, wherein the microfluidic chip comprises a plurality of microchannels configured to form a water-in-oil droplet from a first solution comprising the polyhydroxy compound and/or the antichaotropic agent, a second solution comprising the polyampholyte and a fluid comprising a carrier oil with or without a surfactant.

94. The kit according to claim 90, further comprising a carrier oil supplemented with a surfactant, wherein the surfactant is suitable to stabilize the water-in-oil droplets that are produced during the method of making the microcapsule against coalescence.

95. A thermostable core/shell microcapsule produced by the method of claim 56, said microcapsule comprising:
(a) a core comprising a polyhydroxy compound and/or an antichaotropic agent;
(b) a biological entity such a cell, a microorganism, a bacteria, a virus or a nucleic acid in the core; and
(c) a semi-permeable shell enveloping the core; wherein the said semi-permeable shell is thermostable withstanding temperatures higher than 90° C., and which comprises a covalently cross-linked polyampholyte and decomposes under mild reaction conditions using a protease enzyme.
